SEQUENCE LISTING (38 sequences)

SEQ ID NO: 1 - protein, 7 aa, Artificial Sequence
  Description of Artificial Sequence Synthetic peptide
  Designed peptide to act as the inhibitory loop in a protein
FCRAVKR

SEQ ID NO: 2 - protein, 7 aa, Artificial Sequence
  Description of Artificial Sequence Synthetic peptide
  Designed peptide to act as the inhibitory loop in a protein
YCRAVKR

SEQ ID NO: 3 - protein, 7 aa, Artificial Sequence
  Description of Artificial Sequence Synthetic peptide
  Designed peptide to act as the inhibitory loop in a protein
LCRAVKR

SEQ ID NO: 4 - protein, 7 aa, Artificial Sequence
  Description of Artificial Sequence Synthetic peptide
  Designed peptide to act as the inhibitory loop in a protein
PCRAVKR

SEQ ID NO: 5 - protein, 7 aa, Artificial Sequence
  Description of Artificial Sequence Synthetic peptide
  Designed peptide to act as the inhibitory loop in a protein
QCRAVKR

SEQ ID NO: 6 - protein, 7 aa, Artificial Sequence
  Description of Artificial Sequence Synthetic peptide
  Designed peptide to act as the inhibitory loop in a protein
MCRAVKR

SEQ ID NO: 7 - protein, 7 aa, Artificial Sequence
  Description of Artificial Sequence Synthetic peptide
  Designed peptide to act as the inhibitory loop in a protein
VCRAVKR

SEQ ID NO: 8 - protein, 7 aa, Artificial Sequence
  Description of Artificial Sequence Synthetic peptide
  Designed peptide to act as the inhibitory loop in a protein
WCRAVKR

SEQ ID NO: 9 - protein, 7 aa, Artificial Sequence
  Description of Artificial Sequence Synthetic peptide
  Designed peptide to act as the inhibitory loop in a protein
PCRALKI

SEQ ID NO: 10 - protein, 7 aa, Artificial Sequence
  Description of Artificial Sequence Synthetic peptide
  Designed peptide to act as the inhibitory loop in a protein
PCRAVKI

SEQ ID NO: 11 - protein, 58 aa, Artificial Sequence
  Description of Artificial Sequence Synthetic polypeptide
  Modified Kunitz domain protein
KPDFCFLEEDPGFCRAVKRRYFYNNQTKQCERFKYGGCLGNMNNFETLEECKNICEDG

SEQ ID NO: 12 - protein, 58 aa, Artificial Sequence
  Description of Artificial Sequence Synthetic polypeptide
  Modified Kunitz domain protein
KPDFCFLEEDPGYCRAVKRRYFYNNQTKQCERFKYGGCLGNMNNFETLEECKNICEDG

SEQ ID NO: 13 - protein, 58 aa, Artificial Sequence
  Description of Artificial Sequence Synthetic polypeptide
  Modified Kunitz domain protein
KPDFCFLEEDPGLCRAVKRRYFYNNQTKQCERFKYGGCLGNMNNFETLEECKNICEDG

SEQ ID NO: 14 - protein, 58 aa, Artificial Sequence
  Description of Artificial Sequence Synthetic polypeptide
  Modified Kunitz domain protein
KPDFCFLEEDPGPCRAVKRRYFYNNQTKQCERFKYGGCLGNMNNFETLEECKNICEDG

SEQ ID NO: 15 - protein, 58 aa, Artificial Sequence
  Description of Artificial Sequence Synthetic polypeptide
  Modified Kunitz domain protein
KPDFCFLEEDPGQCRAVKRRYFYNNQTKQCERFKYGGCLGNMNNFETLEECKNICEDG

SEQ ID NO: 16 - protein, 58 aa, Artificial Sequence
  Description of Artificial Sequence Synthetic polypeptide
  Modified Kunitz domain protein
KPDFCFLEEDPGMCRAVKRRYFYNNQTKQCERFKYGGCLGNMNNFETLEECKNICEDG

SEQ ID NO: 17 - protein, 58 aa, Artificial Sequence
  Description of Artificial Sequence Synthetic polypeptide
  Modified Kunitz domain protein
KPDFCFLEEDPGVCRAVKRRYFYNNQTKQCERFKYGGCLGNMNNFETLEECKNICEDG

SEQ ID NO: 18 - protein, 58 aa, Artificial Sequence
  Description of Artificial Sequence Synthetic polypeptide
  Modified Kunitz domain protein
KPDFCFLEEDPGWCRAVKRRYFYNNQTKQCERFKYGGCLGNMNNFETLEECKNICEDG

SEQ ID NO: 19 - protein, 58 aa, Artificial Sequence
  Description of Artificial Sequence Synthetic polypeptide
  Modified Kunitz domain protein
KPDFCFLEEDPGPCRALKIRYFYNNQTKQCERFKYGGCLGNMNNFETLEECKNICEDG

SEQ ID NO: 20 - protein, 58 aa, Artificial Sequence
  Description of Artificial Sequence Synthetic polypeptide
  Modified Kunitz domain protein
KPDFCFLEEDPGPCRAVKIRYFYNNQTKQCERFKYGGCLGNMNNFETLEECKNICEDG

SEQ ID NO: 21 - protein, 58 aa, Unknown
  Description of Unknown Bovidae family sequence
  Bovine pancreatic trypsin inhibitor
RPDFCLEPPYTGPCKARIIRYFYNAKAGLCQTFVYGGCRAKRNMFKSAEDCMRTCGGA

SEQ ID NO: 22 - protein, 58 aa, Artificial Sequence
  Description of Artificial Sequence Synthetic polypeptide
  General sequence of the Kunitz domains
  MOD_RES (1)..(4): Any amino acid or not present
  MOD_RES (6)..(11): Any amino acid
  MOD_RES (13)..(13): Any amino acid
  MOD_RES (15)..(20): Any amino acid
  MOD_RES (21)..(21): Phe, Tyr or Trp
  MOD_RES (22)..(22): Tyr or Phe
  MOD_RES (23)..(23): Tyr or Phe
  MOD_RES (24)..(29): Any amino acid
  MOD_RES (31)..(32): Any amino acid
  MOD_RES (34)..(34): Any amino acid
  MOD_RES (35)..(35): Tyr or Trp
  MOD_RES (36)..(36): Gly or Ser
  MOD_RES (39)..(39): Any amino acid
  MOD_RES (40)..(40): Gly or Ala
  MOD_RES (41)..(42): Any amino acid
  MOD_RES (43)..(43): Asn or Gly
  MOD_RES (44)..(44): Any amino acid
  MOD_RES (45)..(45): Phe or Tyr
  MOD_RES (46)..(50): Any amino acid
  MOD_RES (52)..(54): Any amino acid
  MOD_RES (56)..(58): Any amino acid or not present
XXXXCXXXXXXGXCXXXXXXXXXXXXXXXCXXFXXXGCXXXXXXXXXXXXCXXXCXXX

SEQ ID NO: 23 - protein, 58 aa, Homo sapiens
  Tissue factor pathway inhibitor (UniProt ID P10646) domain 2
KPDFCFLEEDPGICRGYITRYFYNNQTKQCERFKYGGCLGNMNNFETLEECKNICEDG

SEQ ID NO: 24 - DNA, 218 bp, Artificial Sequence
  Description of Artificial Sequence Synthetic polynucleotide
  Codon optimized gene for the 2nd domain of the Tissue Factor Pathway Inhibitor-1 protein, supplied it with the necessary flanking regions for cloning into the display vector between Kpn2I (BspEI) and SacI sites
tccggaggctcgggcaaaccggacttctgcttcctggaagaagacccgggtatctgccgtggttacatcacccgttacttctacaacaaccagaccaaacagtgcgaacgtttcaaatacggtggttgcctgggtaacatgaacaacttcgaaaccctggaagaatgcaaaaacatctgcgaagacggtggcggcagcggcggcagcggcgggagctc

SEQ ID NO: 25 - DNA, 64 bp, Artificial Sequence
  Description of Artificial Sequence Synthetic oligonucleotide
  TFPI D2 P3-P4′ STOP oligonucleotide
cctggaagaagacccgggttaatgctaataataataataacgttacttctacaacaaccagacc

SEQ ID NO: 26 - DNA, 64 bp, Artificial Sequence
  Description of Artificial Sequence Synthetic oligonucleotide
  TFPI D2 P3-P4′ library primer
  modified_base (20)..(21): a, c, t or g
  modified_base (26)..(27): a, c, t or g
  modified_base (29)..(30): a, c, t or g
  modified_base (32)..(33): a, c, t or g
  modified_base (35)..(36): a, c, t or g
  modified_base (38)..(39): a, c, t or g
cctggaagaagacccgggtnnktgcnnknnknnknnknnkcgttacttctacaacaaccagacc

SEQ ID NO: 27 - protein, 390 aa, Rattus sp.
  Catalytic fragment of rat MASP-2 (MASP2cf)
MTQPCPDPTAPPNGHISPVQATYVLKDSFSVFCKTGFELLQGSVPLKSFTAVCQKDGSWDRPIPECSIIDCGPPDDLPNGHVDYITGPEVTTYKAVIQYSCEETFYTMSSNGKYVCEADGFWTSSKGEKSLPVCKPVCGLSTHTSGGRIIGGQPAKPGDFPWQVLLLGETTAAGALIHDDWVLTAAHAVYGKTEAMSSLDIRMGILKRLSPHYTQAWPEAVFIHEGYTHGAGFDNDIALIKLKNKVTINRNIMPICLPRKEAASLMKTDFVGTVAGWGLTQKGFLARNLMFVDIPIVDHQKCATAYTKQPYPGAKVTVNMLCAGLDAGGKDSCRGDSGGALVFLDNETQRWFVGGIVSWGSINCGGSEQYGVYTKVTNYIPWIENIINNF

SEQ ID NO: 28 - DNA, 410 bp, Artificial Sequence
  Description of Artificial Sequence Synthetic polynucleotide
  Synthetic gene
ccatgggccatcaccatcaccatcacgctagcatggcgtcccctctggagaaggccctggatgtgatggtgtccaccttccacaagtactcgggcaaagagggtgacaagttcaagctcaacaagtcagaactaaaggagctgctgacccgggagctgcccagcttcttggggaaaaggacagatgaagctgctttccagaagctgatgagcaacttggacagcaacagggacaacgaggtggacttccaagagtactgtgtcttcctgtcctccatcgccatgatgtctaacgaattctttgaaggcttcccagataagcagcccaggaagaaagtcgacgactacgacatcccgactaccgaaaacctgtacttccagggatcccatatgtatggtacccgactcgag

SEQ ID NO: 29 - DNA, 25 bp, Artificial Sequence
  Description of Artificial Sequence Synthetic oligonucleotide
  TFPI BamHI forward oligonucleotide
agtaggatccaaaccggacttctgc

SEQ ID NO: 30 - DNA, 30 bp, Artificial Sequence
  Description of Artificial Sequence Synthetic oligonucleotide
  TFPI HindIII reverse oligonucleotide
gtataagcttattaaccgtcttcgcagatg

SEQ ID NO: 31 - DNA, 64 bp, Artificial Sequence
  Description of Artificial Sequence Synthetic oligonucleotide
  Mutagenesis primer for the production of the protein according to SEQ ID NO 11
cctggaagaagacccgggtttctgccgtgcggtgaaacgtcgttacttctacaacaaccagacc

SEQ ID NO: 32 - DNA, 64 bp, Artificial Sequence
  Description of Artificial Sequence Synthetic oligonucleotide
  Mutagenesis primer for the production of the protein according to SEQ ID NO 14
cctggaagaagacccgggtccgtgccgtgcggtgaaacgtcgttacttctacaacaaccagacc

SEQ ID NO: 33 - DNA, 25 bp, Artificial Sequence
  Description of Artificial Sequence Synthetic oligonucleotide
  Mutagenesis primer for the production of the protein according to SEQ ID NO 17
cgggtgtgtgccgtgcggtgaaacg

SEQ ID NO: 34 - DNA, 19 bp, Artificial Sequence
  Description of Artificial Sequence Synthetic oligonucleotide
  S100A4 3′ oligonucleotide
ctagtctcgagtgccaagc

SEQ ID NO: 35 - DNA, 21 bp, Artificial Sequence
  Description of Artificial Sequence Synthetic oligonucleotide
  pMal 5′ oligonucleotide
gatgtccgctttctggtatgc

SEQ ID NO: 36 - protein, 38 aa, Artificial Sequence
  Description of Artificial Sequence Synthetic polypeptide
  Synthetic protein of insect origin
GSGEVTCEPGTTFKDKCNTCRCGSDGKSAVCTKLWCNQ

SEQ ID NO: 37 - DNA, 550 bp, Artificial Sequence
  Description of Artificial Sequence Synthetic polynucleotide
  Fusion gene created for the display of TFPI-D2 on the surface of M13 bacteriophage
catatgaaaataaaaacaggtgcacgcatcctcgcattatccgcattaacgacgatgatgttttccgcctcggcgctagcatcggattataaagacgatgatgacaaagcttccggcgggtccggaggctcgggcaaaccggacttctgcttcctggaagaagacccgggtatctgccgtggttacatcacccgttacttctacaacaaccagaccaaacagtgcgaacgtttcaaatacggtggttgcctgggtaacatgaacaacttcgaaaccctggaagaatgcaaaaacatctgcgaagacggtggcggcagcggcggcagcggcgggagctccagcgccgagggtgacgatcccgcaaaagcggcctttaactccctgcaagcctcagcgaccgaatatatcggttatgcgtgggcgatggttgttgtcattgtcggcgcaactatcggtatcaagctgtttaagaaattcacctcgaaagcaagctaataatctagagcggccgccccgcggtggagcttgaattcgccctatagtgagtcgt

SEQ ID NO: 38 - protein, 164 aa, Artificial Sequence
  Description of Artificial Sequence Synthetic polypeptide
  Fusion protein created for the display of TFPI- D2 on the surface of M13 bacteriophage
HMKIKTGARILALSALTTMMFSASALASDYKDDDDKASGGSGGSGKPDFCFLEEDPGICRGYITRYFYNNQTKQCERFKYGGCLGNMNNFETLEECKNICEDGGGSGGSGGSSSAEGDDPAKAAFNSLQASATEYIGYAWAMVVVIVGATIGIKLFKKFTSKAS

(19) United States
(12) Patent Application Publication
(10) Pub. No.: US 2020/0131249 A1
(43) Pub. Date: Apr. 30, 2020

(54) NOVEL PROTEINS AND USE THEREOF

(71) Applicant: EVOLVERITAS KFT., Szentes (HU)

(72) Inventors: Peter GAL, Budapest (HU); Gabor PAL, Budapest (HU); David SZAKACS, Budapest (HU)

(73) Assignee: EVOLVERITAS KFT., Szentes (HU)

(21) Appl. No.: 16/476,443

(22) PCT Filed: Jan. 4, 2018
(86) PCT No.: PCT/HU2018/050001

§ 371 Date: Jul. 8, 2019

(30) Foreign Application Priority Data
Jan. 6, 2017 (HU) P1700012

(51) Int. Cl. C07K 14/81; G01N 33/68

(52) U.S. Cl. CPC C07K 14/8114; G01N 33/68; A61K 38/00; C07K 2319/43; C07K 2319/034

(57) ABSTRACT

The invention relates to proteins containing any of the sequences according to general formula (Ih):

X₁CRX₂X₃X₄X₅ (Ih)

where
X₁ is F, Y, L, P, Q, M, V, W, A, T and
X₂ is A, G, S, T and
X₃ is V, A, I, L, M, D, H, S and
X₄ is K, I, Q, R, H, S, F, M, N, L, V and
X₅ is R, V, I, K, M, Q, E, F, L, N, Y, D, S, H. and
its salts, esters and pharmaceutically acceptable prodrugs. The invention further relates to pharmaceutical preparations and kits containing said proteins, to screening procedures using said proteins. The proteins of the invention are useful in the treatment or prevention of diseases that can be treated by inhibiting the complement system.

### FIELD OF THE INVENTION

The invention relates to novel proteins, and it also relates to a process for the production of such proteins and to the use of such proteins in the production of medicines.

### BACKGROUND OF THE INVENTION

The complement system is one of the most important components of innate immunity in humans and in all vertebrates including mammalians. The complement system, as the immune system in general, is able to recognise, label and remove intruding pathogens and dangerously altered host structures (e.g. apoptotic cells). There are different mechanisms through which the complement system recognizes the pathogen-associated molecular patterns (PAMPs) and the damage- or danger-associated molecular patterns (DAMPs). The complement system, as a major effector arm of the innate immune system, forms one of the first defence lines of the organism against pathogenic microorganisms, but it also links to the adaptive (acquired) immune system at several points forming a bridge between innate and adaptive immune mechanisms (Walport 2001a; Walport 2001b; Morgan 2005; Ricklin 2010; Merle 2015a; Merle 2015b). The complement system is a network consisting of about 30 protein components, which components can be found in the blood plasma in soluble form, and also in the form of receptors and modulators (e.g. inhibitors) attached to the surface of cells. The main components of the system are serine protease zymogens, which activate each other in a cascade-like manner in strictly determined order. Certain substrates of the activated proteases are proteins containing a thioester bond (components C4 and C3 in the complement system). When these substrates are cleaved by the activated proteases, the reactive thioester group becomes exposed on the surface of the molecule, and in this way, it is able to attach the cleaved molecule to the surface of the attacked cell. As a result of this, such cells are labelled so that they can be recognised by the immune system.

The biological functions of the complement system are extremely diverse and complex, and up till now they have not been explored in every detail. One of the most important functions is direct cytotoxic activity, which is triggered by the membrane attack complex (MAC) formed from the terminal components of the complement system. The MAC perforates the membrane of cells recognised as foreign, which results in the lysis and, thereby, destruction of such cells. This defence mechanism is very important against Gram-negative bacteria (mostly Neisseria species) (Petersen 1996, Lewis 2014).

Another important function of the complement system is opsonisation, when complement components (e.g. C1q, MBL, C4b, C3b) settle on the surface of the cells being recognised as foreign or dangerously altered self, and promote the phagocytosis by leukocytes (e.g. macrophages). These leukocytes then engulf the cells to be destroyed.

Furthermore, the inflammation initiation role of the complement system is also important. The cleavage products released during complement activation initiate an inflammatory process through their chemotactic stimulating effects on leukocytes (neutrophils, monocytes and macrophages) (Mollnes 2002).

Complement components C3a and C5a are the most potent anaphylatoxins that exert their proinflammatory effects through G-protein-coupled receptors (C3aR, C5aR).

The components of the complement system are present in blood plasma in an inactive (zymogenic) form until the activation of the complement cascade is triggered by an appropriate signal (e.g. intrusion of a foreign cell, pathogen). The normal activity of the complement system is important from the aspect of maintaining immune homeostasis. Both its abnormal underactivity and its uncontrolled hyperactivity may result in the development of severe diseases or in the aggravation of already existing diseases (Szebeni 2004).

The complement system can be activated via three different pathways: the classical pathway, the lectin pathway and the alternative pathway.

In the first step of the classical pathway the C1 complex binds to the surface of the activator, that is the biological structure recognised as foreign. The C1 complex is a supramolecular complex consisting of a recognition protein molecule (C1q) and serine proteases (C1r, C1s) associated to it (Arlaud 2002). First of all, the C1q molecule binds to immune complexes, apoptotic cells, C-reactive protein or to other activator structures. As a result of the C1q molecule binding to the activator, the serine protease zymogens present in the C1 complex become gradually activated. In the tetramer C1s-C1r-C1r-C1s first the C1r zymogens autoactivate, then the active C1r molecules cleave and activate the C1s molecules. The active C1s cleaves the C4 and C2 components of the complement system, which cleavage products are the precursors of the C3-convertase enzyme complex (C4bC2a). The C3-convertase splits C3 molecules into C3a and C3b fragments, and the depositing C3b molecules on the activator surface initiate the alternative pathway. As the density of the deposited C3b on the activator surface is increasing the C3-convertase transforms into C5-convertase (C4bC2aC3b). The C5-convertase cleaves C5, after which the activation of the complement system culminates in the terminal phase (formation of the MAC, also known as the terminal complement complex TCC) characteristic of all three pathways. The activation of the lectin pathway, a different pathway of the complement system, is similar to that of the classical pathway (Fujita 2004). However, in this pathway several different types of recognition molecules are involved: MBL (“mannose-binding lectin”), ficolins (ficolin-1, ficolin-2 and ficolin-3, also known as M, L and H ficolin types, respectively) and collectins (collectin liver 1=CL-L1, collectin kidney 1=CL-K1, collectin placenta 1=CL-P1). These molecules bind to the carbohydrate structures on the surface of microorganisms. The binding of these recognition molecules is followed by the autoactivation of MASP-1 (“MBL-associated serine protease”-1) zymogen. Activated MASP-1 then cleaves and activates zymogen MASP-2. It has been demonstrated that MASP-1 is the exclusive activator of MASP-2 in normal human blood (Héja 2012a; Degn 2012). The activated MASP-2 cleaves the C4 and C2 components, which results in the formation of the C3-convertase enzyme complex already described in the course of the classical pathway, and from this point the process continues as described above.

The alternative pathway starts with the cleavage of the C3 component into C3a and C3b (see above). C3b anchors to the surface of the biological structure recognised as foreign (Harboe 2008). If the C3b component created during the cleavage is bound to the cell membrane of a microorganism, then it can also bind the zymogenic form of a serine protease called factor B, thereby generating the C3bB proconvertase complex. The zymogen factor B component of this complex is then cleaved and thereby activated by factor D, a complement factor that is present in the blood in active form. The active C3bBb complex created in this way is the C3-convertase of the alternative pathway, which, after being completed with a further C3b molecule, transforms into a C5 convertase. The alternative pathway may also be triggered spontaneously, independently of the other two pathways by the slow hydrolysis of the C3 component (C3(H₂O)), but if either the classical or the lectin pathway gets to the point of C3 cleavage, the alternative pathway significantly amplifies their effect.

Of the three pathways introduced above, we describe the lectin pathway in greater detail, as this most recently discovered complement pathway is also the most important one from the aspect of the present invention. The lectin pathway has many different recognition molecules (MBL of different degrees of polymerisation, ficolins, collectins) and all recognition molecules bind several different types of proteases and non-catalytic proteins. MASP-2 even in itself is able to initiate the complement cascade in vitro at relatively high concentrations (Ambrus 2003; Gál 2005). In vivo, however, the autoactivation potential of MASP-2 does not manifest in normal human blood, where this enzyme is present at a low concentration (about 0.5 μg/ml). In these conditions, the activation of MASP-2 strictly depends on the MASP-1 mediated cleavage of the zymogen. The physiological function of the MASP-1 protease, which is present at a higher concentration (7-10 μg/ml), has not been completely explored yet, but it has been unambiguously demonstrated that it is the exclusive activator of MASP-2 in normal human blood (or serum or plasma) (Héja 2012a).

MASP-2 is the only protease in the lectin pathway which can cleave C4 therefore it is absolutely necessary for C3 convertase (C4b2a) generation. MASP-1 on its own is not able to initiate the complement cascade (it can cleave C2 but not C4), but it contributes significantly to the C3 convertase generation via the cleavage of C2 (Héja 2012a). Several signs indicate that to a certain extent MASP-1 is a protease similar to thrombin, forming a bridge between the two major proteolytic cascade systems in the blood: the complement system and the blood coagulation system (Hajela 2002; Krarup 2008, Megyeri 2009, La Bonte 2012).

Both the MASP-1 and the MASP-2 genes have alternative splice products. The MAp19 (MAP-2 or sMAP) protein containing the first two domains of MASP-2 (CUB1-EGF) is produced from the MASP-2 gene. The MAp44 (MAP-1) and the MASP-3 mRNA are transcribed from the MASP-1 gene. Similarly to the MAp19, the MAp44 is also a truncated protein: it contains the first four non-catalytic domains of the MASP-1 protein (CUB1-EGF-CUB2-CCP1), consequently it does not have proteolytic activity (Degn 2009). Its function is unknown, but it probably plays a role in regulation. The first five domains of MASP-3 are the same as those of MASP-1, but the two proteins differ in their serine protease domain. MASP-3 has low catalytic activity on synthetic substrates. MASP-3 cannot autoactivate, but it can be cleaved by MASP-1 and MASP-2 in vitro. It has been recently demonstrated that MASP-3 is solely responsible for the conversion of pro-factor D into active factor D through limited proteolysis in resting normal human blood (Dobó 2016). In this way, the alternative and lectin pathways are fundamentally linked. Unlike other early proteases, MASP-3 does not form a complex with the C1-inhibitor molecule. The presence of MAp19, MAp44 and MASP-3 presumably acts against the activation of the lectin pathway, as these proteins compete with the active MASP-2 and MASP-1 enzymes for the binding sites on the recognition molecules.

As it has been mentioned above, abnormal operation of the complement system in the human or animal organism may result in developing disease. The uncontrolled activation of the complement system may result in damaging self-tissues, and developing inflammatory or autoimmune conditions (Beinrohr 2008).

One of these conditions is ischemia-reperfusion (hereinafter: IR) injury, which occurs, when the oxygen supply of a tissue is temporarily restricted or interrupted (ischemia) for any reason (e.g. vascular obstruction), and after the restoration of blood circulation (reperfusion) cellular destruction starts. During reperfusion, the complement system recognises ischemic cells as altered self-cells and starts an inflammatory reaction to remove them. Partly this phenomenon is responsible for tissue damage occurring after myocardial infarction and stroke, and it may also cause complications during coronary bypass surgery and organ transplantations (Markiewski 2007). Several research groups reported that the lectin pathway plays a role in the development of IR injury. This suggests that deliberate suppression of the lectin pathway should reduce the extent and the consequences of IR injury. In fact, it was shown that inhibition of MASP-2 is an efficient method to reduce the IR injury. In a mouse model, it was demonstrated that targeting (abolishing or blocking) of MASP-2 confers protection from myocardial and gastrointestinal IR injury (Schwaeble 2012). While this protection is MASP-2 dependent, in this mouse model it was found that C4 did not play a role in the MASP-2-mediated IR injury. Similarly, in the case of renal IR injury the tissue damage was MASP-2 dependent, but C4 independent in the mouse model (Asgari 2014). The natural, endogen lectin pathway inhibitor (MAp44) was also effective in attenuating myocardial IR injury (Pavlov 2012). MAp44, as a non-catalytic fragment of MASP-1/3 is capable of displacing MASP-1 and MASP-2 from the pattern recognition molecules. In a mouse model, where the animal expresses human MBL, anti-MBL antibodies reduced the size of myocardial IR injury (Pavlov 2015). The protecting effect of lectin pathway inhibition was also demonstrated in a renal IR model in pigs, as well (Castellano 2010). Furthermore, targeting MASP-2 in mice mediated protection against post-ischemic brain injury, as well (Orsini 2016). In line with this preclinical finding, smaller infarction size and better functional outcomes were revealed also in MBL deficient patients after ischemic stroke (Osthoff 2011). In summary, the above described results strongly suggest that inhibition of the lectin pathway and particularly inhibition of the MASP-2 enzyme can prevent or alleviate IR injuries of different organ systems in various animal models as well as in human patients.

The involvement of lectin pathway activation has also been implicated in the pathogenesis of rheumatoid arthritis (Ammiztboll 2012) and also in juvenile idiopathic arthritis (Petri 2015).

Excessive activity of the complement system also plays a role in the development and maintenance of various neurodegenerative diseases (e.g. Alzheimer's, Huntington's and Parkinson's diseases and Multiple Sclerosis) (Tichaczek-Goska 2012; Ingram 2009).

Uncontrolled activity of the complement system is one of the main factors in the pathogenesis of age-related macular degeneration (AMD) as well (Bora 2008), a disorder responsible for half of all cases of age-related loss of eyesight in developed industrial countries.

Enhanced activation of the complement system can also be associated with one of the forms of autoimmune nephritis, i.e. C3 glomerulopathy (with appearance forms of “dense deposit disease” (DDD) or “C3 glomerulonephritis”) and with another autoimmune disease, namely systemic lupus erythematosus (SLE) (Cook 2013). Progressive damage of the glomeruli in DDD can lead to severe (frequently end-stage) kidney failure.

Atypical hemolytic uremic syndrome (aHUS) is a complement-related disease manifesting in microangiopathic hemolytic anemia, thrombocytopenia, vascular damage with thrombosis, and organ injury, typically that of the kidney (Noris 2009). The complement system attacks the kidney endothelium promoting the formation of microthrombi in the renal microvasculature. The development of aHUS is associated with uncontrolled complement activation due to mutations in complement regulatory proteins (e.g. factor H).

Hemolytic uremic syndrome (HUS) can also be elicited by bacterial infections. Bacterial toxins (e.g. Shiga-toxin) compromise the regulation of the complement cascade resulting in uncontrolled activation of the complement system (Conway 2015) Inhibition of the lectin pathway of complement activation provided protection against HUS in a mouse model of HUS (Ozaki 2016).

The intravenous administration of certain diagnostic agents, especially when they are used in liposomes, may generate allergic type reactions independent from antibodies in patients. This pseudo-allergic reaction is due to the activation of the complement system (complement activation-related pseudo-allergy, abbreviated as CARPA) (Szebeni 2005). The released inflammation initiating substances (e.g. C5a, C3a) mobilise the cellular elements of the immune system. With the selective inhibition of the complement system fatal pseudo-allergic reactions can be suppressed.

Uncontrolled activation of the complement system also plays a role in paroxysmal nocturnal hemoglobinuria (PNH) and the disease can be alleviated by inhibition of the complement cascade (Kelly 2011).

Inhibition deficiency of the lectin pathway of complement activation may also mediate protection against graft rejection after organ transplantation (Fildes 2008; Ibernon 2014).

Excessive activation of the complement cascade implicating potential therapeutic role for complement activation inhibitors has been demonstrated in polytrauma patients (Burk 2012).

If the complement system is inhibited at the level of the first, pathway-specific activation steps, the efficient and selective inhibition of individual activation pathways becomes possible without triggering general immunosuppression. Thus, in order to treat the above mentioned diseases, the lectin pathway can be blocked selectively by inhibiting the MASP-2 enzyme, while the classical pathway responsible for the elimination of immunocomplexes is left untouched and remains fully functional.

The C1r, C1s, MASP-1, MASP-2 and MASP-3 enzymes form an enzyme family having the same domain structure (Gál 2007). The trypsin-like serine protease (SP) domain responsible for proteolytic activity is preceded by five non-catalytic domains. The three domains CUB1-EGF-CUB2 forming the N-terminal part of the molecule (CUB=C1r/C1s, sea urchin Uegf and Bone morphogenetic protein-1; EGF=Epidermal Growth Factor) are responsible for the dimerisation of the molecules (both in the case of MASP-1 and MASP-2) and for interacting with other molecules, e.g. for binding to the recognition molecules.

In respect of catalytic properties, the C-terminal CCP1-CCP2-SP fragment (CCP=Complement Control Protein) of the molecules is functionally equivalent with the full-length molecule. One of the characteristic features of complement proteases is that they have very narrow substrate specificity: they are able to cleave the well-defined peptide bonds of only a few protein substrates. Both the CCP modules and the SP domain contribute to this finely tuned specificity. It has been shown that the CCP domains of MASP-2 contain exosite for the C4 substrate (Kidmose 2012).

The SP domain contains the active centre characteristic of serine proteases, the substrate binding pocket and the oxyanion hole. Eight surface loop regions, the conformation of which is quite different in the different proteases, play a decisive role in determining subsite specificity. On the one part, the CCP modules stabilise the structure of the catalytic region, and on the other part they contain binding sites for large protein substrates.

As outlined above, selective inhibition of early members of the lectin pathway could lead to the effective treatment of some diseases.

Although the small-molecule compounds generally used for inhibiting trypsin-like serine proteases (e.g. benzamidine, NPGB, FUT-175) inhibit the activity of complement proteases too (Schwertz 2008), this inhibition is not selective enough; it also extends to the inactivation of other serine proteases in the blood plasma, e.g. blood coagulation enzymes and kallikreins.

The natural inhibitors of the early complement proteases are typically serpins. C1 inhibitor inhibits the classical and the lectin pathway as well. It makes a stable covalent acyl-enzyme complex with C1r, C1s, MASP-1 and MASP-2. The C1 inhibitor protein circulating in blood and belonging to the serpin family is also characterised by relatively broad specificity. It also inhibits proteases of the blood coagulation (FXIa and FXIIa), and plasma kallikrein. Another serpin, antithrombin was also shown to inhibit MASP-1 and MASP-2, consequently the lectin pathway. In the presence of heparin antithrombin is as efficient inhibitor of the lectin pathway as the C1 inhibitor (Paréj 2013). The blood borne canonical inhibitor TFPI (tissue factor pathway inhibitor) is a very weak inhibitor of MASP-2 (Keizer 2015).

International patent application Pub. No. WO 2010/136831 discloses oligopeptides that are inhibitors of the MASP enzymes, selectively inhibiting the lectin pathway. Some of them were selective MASP-2 inhibitors over the MASP-1 enzyme while some of them were not selective between MASP-1 and MASP-2. The oligopeptides described in this prior art are of plant origin, i.e. these peptides were evolved by the phage display technique from the 14-amino acid length Sun Flower Trypsin Inhibitor (SFTI) and termed as SFTI-based MASP Inhibitors (SFMI) (Kocsis 2010).

International patent application Pub. No. WO 2012/007777 discloses proteins that have certain MASP inhibitory sequence. These sequences were evolved also by the phage display technique starting from the sequence of the inhibitory loop of the S. gregaria Chymotrypsin Inhibitor (SGCI). The MASP inhibitors described in this prior art and termed as SGCI-based MASP Inhibitors (SGMI) are of insect origin and are selective either for MASP-1 or for MASP-2 (Héja 2012b).

International patent application Pub. No. WO 2011/047346 discloses MASP-2 inhibitors for the treatment of complement mediated coagulation disorder.

Inhibitory oligopeptides are often inserted in proteins to keep the functional structure of the peptide intact and to prevent decomposition by proteases or by other factors. A generally used choice of this kind of host proteins is the protease inhibitor Kunitz domain. Kunitz domain type proteins are widely used for this purpose as they are stable and easy to produce. Such modified Kunitz domains are useful biopharmaceuticals acting as specific protease inhibitors. U.S. Pat. No. 5,994,125 discloses Kunitz domain type proteins that inhibit the serine protease human plasma kallikrein. Definition, features and use of Kunitz domains are described in U.S. Pat. No. 5,994,125, which is therefore hereby incorporated by reference in its entirety.

### SUMMARY OF THE INVENTION

The inhibition of the complement system, including the lectin pathway, may be an efficient tool in fighting against human diseases occurring as a result of the abnormal activity of the complement system. The presently known inhibitors have either the plant-originated SFTI peptide structure (see WO 2010/136831, SFTI-based SFMI inhibitors are described) or have the insect-originated Pacifastin protein structure (see WO 2012/007777, where Pacifastin-based SGMI inhibitors are described). These SFTI-based SFMI inhibitors can either inhibit both proteases (MASP-1 and MASP-2) or are selective MASP-2 inhibitors, while among the above mentioned Pacifastin-based SGMI inhibitors there are both MASP-1 specific and MASP-2 specific inhibitors.

Both the plant origin and the insect origin MASP inhibitors block the activation of the human lectin pathway, but these non-human compounds impose a significant risk of immunogenicity in the human host.

For this reason, we set the aim to develop efficient and selective MASP-2 inhibitor compounds that are based on a human protein and therefore impose a significantly lower risk of immunogenicity in humans.

We surprisingly found that the human protein based compounds having the sequence of general formula (Ih) meet the objective of the present invention, i.e. they are efficient and selective inhibitors of the human MASP-2 enzyme.

The proteins according to the present invention are preferably based on the Kunitz-type scaffold of the second domain (residues 121-178) of the human Tissue Factor Pathway Inhibitor-1 protein (TFPI-1; UniProt ID P10646), hereafter referred to as TFPI-D2.

Surprisingly we found that the proteins containing any of the following sequences according to general formula (Ih) are suitable for inhibiting human MASP-2:

X₁CRX₂X₃X₄X₅ (Ih)

where

### X₁ is F, Y, L, P, Q, M, V, W, A, T and

### X₂ is A, G, S, T and

### X₃ is V, A, I, L, M, D, H, S and

### X₄ is K, I, Q, R, H, S, F, M, N, L, V and

### X₅ is R, V, I, K, M, Q, E, F, L, N, Y, D, S, H.

In accordance with the above outlined objective of the present invention, the term general formula (Ih) defines the set of amino acid sequences that shall be present in a protein that inhibit human MASP-2 enzyme.

In accordance with the above, the invention relates to proteins containing any of the sequences according to general formula (Ih), their salts, esters and pharmaceutically acceptable prodrugs.

More preferably, the invention relates to proteins containing any of the following sequences:

(SEQ ID NO: 1)
FCRAVKR,
(SEQ ID NO: 2)
YCRAVKR,
(SEQ ID NO: 3)
LCRAVKR,
(SEQ ID NO: 4)
PCRAVKR,
(SEQ ID NO: 5)
QCRAVKR,
(SEQ ID NO: 6)
MCRAVKR,
(SEQ ID NO: 7)
VCRAVKR,
(SEQ ID NO: 8)
WCRAVKR,
(SEQ ID NO: 9)
PCRALKI
(SEQ ID NO: 10)
PCRAVKI

and their salts, esters or prodrugs.

Most preferably the invention relates to proteins containing any of the following sequences:

(SEQ ID NO: 1)
FCRAVKR,
(SEQ ID NO: 4)
PCRAVKR,

and their salts, esters, or prodrugs.

The invention preferably relates to Kunitz domain proteins, where the sequence tag from position 13 to position 19 according to the position numbering defined for the Kunitz domain in SEQ ID NO: 22 has any of the sequences of the general formula (Ih), more preferably any of the sequences from SEQ ID NO: 1 to SEQ ID NO: 10.

The Kunitz domain protein is preferably a modified TFPI-D2 protein.

The present invention preferably relates to proteins which are sequentially analogous to the sequences from SEQ ID NO: 11 to SEQ ID NO: 20.

Sequences from SEQ ID NO: 11 to SEQ ID NO: 20 are the following:

KPDFCFLEEDPGXXXXXXXRYFYNNQTKQCERFKYGGCLGNMNNFETLEEC
KNICEDG,

where XXXXXXX in the amino acid sequence denotes any of the sequences of SEQ IDs NO 1 to 10.

The invention preferably relates to proteins selected from the sequences from SEQ ID NO: 11 to SEQ ID NO: 20.

Furthermore, the invention also relates to pharmaceutical preparations that contain at least one protein containing a sequence according to the general formula (Ih), its salt, ester or prodrug and at least one further additive. This additive is preferably a matrix ensuring controlled active agent release.

The invention relates especially to pharmaceutical preparations that contain at least one of the proteins containing any of the following sequences:

(SEQ ID NO: 1)
FCRAVKR,
(SEQ ID NO: 2)
YCRAVKR,
(SEQ ID NO: 3)
LCRAVKR,
(SEQ ID NO: 4)
PCRAVKR,
(SEQ ID NO: 5)
QCRAVKR,
(SEQ ID NO: 6)
MCRAVKR,
(SEQ ID NO: 7)
VCRAVKR,
(SEQ ID NO: 8)
WCRAVKR,
(SEQ ID NO: 9)
PCRALKI
(SEQ ID NO: 10)
PCRAVKI

and/or their pharmaceutically acceptable salts, esters, and prodrugs.

The invention most preferably relates to pharmaceutical preparations that contain at least one of the proteins containing any of the following sequences:

(SEQ ID NO: 1)
FCRAVKR,
(SEQ ID NO: 4)
PCRAVKR,

and/or their pharmaceutically acceptable salts, esters, and prodrugs.

The pharmaceutical preparations according to the present invention are preferably in the form of infusions, tablets, powders, granules, suppositories, injections, syrups, and intranasal delivery systems.

The invention further relates to kits containing at least one protein containing a sequence according to general formula (Ih), its salt or ester.

The invention also relates to the screening procedure of compounds potentially inhibiting the human MASP-2 enzyme, in the course of which i) a protein according to the general formula (Ih) in a labelled form is added to a solution containing said human MASP-2, then ii) the solution containing one or more compounds to be tested is added to it, and iii) the amount of the released labelled protein is measured.

The invention also relates to the use of proteins containing a sequence according to general formula (Ih) and their pharmaceutically acceptable salts, esters or prodrugs in the production of a pharmaceutical preparation suitable for the treatment or prevention of diseases that can be treated by inhibiting the complement system. In accordance with this, diseases can be selected preferably from the following non-limiting groups:

(1) ischemia-reperfusion (IR) injuries (especially following recanalyzation after arterial occlusion due to thrombosis or other obstructive diseases), including those occurring after myocardial infarction (e.g. treated by percutaneous coronary interventions or thrombolysis), coronary bypass surgery, organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), rheumatoid arthritis (RA), juvenile idiopathic arthritis, age-related macular degeneration, systemic lupus erythematosus (SLE), atypical hemolytic uremic syndrome (aHUS), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; (3) neurodegenerative diseases, preferably Alzheimer's disease, Huntington's disease, Parkinson's disease and multiple sclerosis.

The proteins according to the present invention are useful in the treatment of the above diseases.

The invention also relates to a procedure for isolating the human MASP-2 enzyme, in the course of which i) a carrier with one or more immobilised proteins with sequences according to general formula (Ih) are contacted with a solution containing said human MASP-2 enzyme and ii) the preparation is washed.

### SHORT DESCRIPTION OF THE DRAWINGS

In the drawings

FIG. 1 shows a schematic representation of the phage display method used for evolving the inhibitors of the present invention;

FIG. 2 shows the DNA and amino acid sequence of the fusion gene created for the display of TFPI-D2 on the surface of M13 bacteriophage

FIG. 3 shows the sequence logo diagrams of the obtained sequences, where

FIG. 3.a shows the sequence diagram relating to the sequences selected on the human MASP-2 enzyme;

FIG. 3.b shows the sequence diagram relating to the sequences selected on the rat MASP-2 enzyme;

FIG. 4 shows the conservation pattern observed within the Kunitz family on the example of TFPI-D2 as a member of the Kunitz family.

### DETAILED DESCRIPTION OF THE INVENTION

Although the present invention relates to human MASP-2 inhibitors, the way towards the invention included the research on rat MASP-2 inhibitors, as well. The latter research part aimed to reveal, which human MASP-2 inhibitors could also inhibit rat MASP-2. These bispecifc inhibitors are useful for in vivo studies performed in rat as animal model. The research work can not be divided into “human” and “rat” parts. Nevertheless, besides describing the development of human MASP-2 inhibitors, the description below also contains references to the development of rat MASP-2 inhibitors. The sole purpose of the rat MASP-2 related information is to provide a full support to the invention.

If “sequence” is mentioned in the present description without a prefix of “amino acid” or “nucleic acid”, an amino acid sequence shall be understood.

General formula (Ih) describes amino acid sequences using the one-letter code of amino acid residues known by a person skilled in the art. The positions of the seven unit long sequences are denoted by X₁ to X₅ in case the amino acid at said position is variable, and are denoted by a certain one-letter code (e.g. C or R) if it is constant. The possibilities in positions X₁ to X₅ are shown with the one-letter codes. For example, if in case of general formula (Ih) X₂ is said to be A, G, S, T, it means that alanine, glycine, serine and threonine may be the choice in position X₂. We used the IUPAC recommendations to mark the amino acid side chains in the given sequences (Nomenclature of α-Amino Acids, Recommendations, 1974—Biochemistry, 14(2), 1975).

The present invention relates to proteins that have a loop sequence according to any of sequences of the general formula (Ih) inhibiting human MASP-2. The preferred sequences for this loop sequence are shown in SEQ ID NO: 1 to SEQ ID NO: 10, where SEQ ID NO: 1 and SEQ ID NO: 4 are the most preferred ones. The person skilled in the art understands that the sequence of said loop is the essence of the present invention. This loop is mentioned throughout the present specification as inhibitory loop. Other parts of the protein are for providing the necessary molecular environment of the loop, i.e. stabilize the effective spatial arrangement of the atoms, that is the conformation of the loop. Further functions of the protein parts beyond said loop can be for example:
providing the necessary solubility of the protein in a drug product;
carrying other molecular objects, like labels, anchoring elements;
providing beneficial pharmacokinetic and pharmacodynamic properties for the drug product.

The person skilled in the art will understand that for ensuring these functions of the protein further molecular elements may be needed, like e.g. further amino acid sequence extensions on any of the end parts, modified amino acids, carbohydrate moieties, specific small molecular or biomolecular compounds, etc. Keeping the above mentioned essence of invention in mind, such kind of modified proteins are also within the scope of the present invention. The term host protein is used throughout the present description to refer to proteins into which any of the sequences of the general formula (Ih) is inserted to form the proteins of the present invention.

The present invention relates to proteins and protein derivatives selectively inhibiting the human MASP-2 enzyme. By selective inhibition we understand first of all a selectivity over MASP-1 and MASP-3 enzymes. It will be obvious for a person skilled in the art that the protein environment of the sequences of the general formula (Ih) may influence the successful inhibition.

The present invention also relates to proteins and protein derivatives which are sequentially analogous to the described sequences SEQ ID NO: 11 to SEQ ID NO: 20 and the biological activity of which is also analogous when compared to these described sequences. A person skilled in the art finds it obvious that certain side chain modifications or amino acid replacements can be performed without altering the biological function of the protein in question. Such modifications may be based on the relative similarity of the amino acid side chains, for example on similarities in size, charge, hydrophobicity, hydrophilicity, etc. The aim of such changes may be to increase the stability of the protein against enzymatic decomposition or to improve certain pharmacokinetic or other parameters.

The scope of protection of the present invention also includes proteins into which elements ensuring detectability (e.g. fluorescent group, radioactive atom, etc.) are integrated. This kind of labelling is useful according to the state of the art in diagnostic methods, research works etc.

Furthermore, the scope of protection of the present invention also includes proteins that contain a few further amino acids at their N-terminal, C-terminal, or both ends, additional to what is shown in SEQ ID NO: 11 to SEQ ID NO: 20, and SEQ ID NO 22 if these further amino acids do not have a significant influence on the biological activity of the original sequence. The aim of such further amino acids positioned at the ends may be to facilitate immobilisation, ensure the possibility of linking to other reagents, influence solubility, absorption and other characteristics.

Under Kunitz family or Kunitz domain the following shall be understood within the scope of the present invention. The above referred published patent application No. U.S. Pat. No. 5,994,125 A gives a detailed description of the Kunitz domain. Briefly, Kunitz domain means a homologue of bovine pancreatic trypsin inhibitor, hereinafter BPTI (not of the Kunitz soya-bean trypsin inhibitor). A Kunitz Domain is a domain of a protein having at least 51 amino acids (and up to about 61 amino acids) containing at least two, and preferably three, disulfides. Herein, the residues of all Kunitz domains are numbered as 1-58 by reference to the 58 aminoacid residue mature form of BPTI, amino-acid sequence of SEQ ID NO: 21. Note that the full-length, prepro form of BPTI contains 100 aminoacid residues, and the 58-residue matured segment corresponds to the segment of 36-93 according to the full-length numbering. We note here that the sequence of mature BPTI disclosed in Table 2 of U.S. Pat. No. 5,994,125 as SEQ ID NO: 2, contains a Met in (matured) position 44, while in several published BPTI sequences there is an Asn in this position (see e.g. Uniprot P00974, residue 79 according to the full-length numbering). However, this difference does not influence the definition of the Kunitz domain from the point of view of our invention. Thus, the first cysteine residue is residue 5 and the last cysteine is residue 55. An amino-acid sequence shall, for the purposes of this invention, be deemed a Kunitz domain if it can be aligned, with three or fewer mismatches, to the sequence of SEQ ID NO: 22. An insertion or deletion of one residue shall count as one mismatch. In SEQ ID NO: 22, “x” matches any amino acid and “X” matches the types listed for that position. Disulfide bonds link at least two of: 5 to 55, 14 to 38, and 30 to 51. The number of disulfides may be reduced by one, but none of the standard cysteines shall be left unpaired. Thus, if one cysteine is changed, then a compensating cysteine is added in a suitable location or the matching cysteine is also replaced by a non-cysteine (the latter being generally preferred). For example, Drosophila funebris male accessory gland protease inhibitor has no cysteine at position 5, but has a cysteine at position −1 (just before position 1); presumably this forms a disulfide to Cys55. If Cys14 and Cys18 are replaced, the requirement of Gly12, (Gly or Ser)37, and Gly36 are dropped. From zero to many residues, including additional domains (including other Kunitz Domains), can be attached to either end of a Kunitz domain.

The general sequence of the Kunitz domains is as follows (SEQ ID NO: 22):

xxxxCxxxxxxGxCxxxxxxXXXxxxxxxCxxFxXXGCxXxxXxXxxxxxCxxxCxxx

where:
x1 to x4, x58, x57, and x56 may be variable or absent,
x6 to x11, x13, x15 to x20, x24 to x29, x31 to x32, x34, x39, x41 to x42, x44, x46 to x50, x52 to x54 may be variable,

### X21=Phe, Tyr, Trp,

### X22=Tyr or Phe,

### X23=Tyr or Phe,

### X35=Tyr or Trp,

### X36=Gly or Ser,

### X40=Gly or Ala,

### X43=Asn or Gly, and

### X45=Phe or Tyr

As outlined above the present invention preferably relates to Kunitz domain proteins, where the sequence tag from position 13 to position 19 according to the position numbering defined above for the Kunitz domain in SEQ ID NO: 22 has any of the sequences of the general formula (Ih), more preferably sequences from SEQ ID NO: 1 to SEQ ID NO: 10. The Kunitz domain protein is preferably the TFPI-D2 protein modified in such a way that it contains any of the sequences of the general formula (Ih), more preferably sequences from SEQ ID NO: 1 to SEQ ID NO: 10.

The most preferred proteins according to the present invention have the amino acid sequences selected from the following group: SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19 and SEQ ID NO: 20. These proteins were derived from the host protein TFPI-D2 protein within the Kunitz family.

The present invention also relates to the pharmaceutically acceptable salts of the proteins containing sequences according to general formula (Ih) according to the invention. By this we mean salts, which, during contact with the corresponding human tissues, do not result in an unnecessary degree of toxicity, irritation, allergic symptoms or similar phenomena. As non-restrictive examples of acid addition salts, the following are mentioned: acetate, citrate, aspartate, benzoate, benzene sulphonate, butyrate, digluconate, hemisulphate, fumarate, hydrochloride, hydrobromide, hydroiodide, lactate, maleate, methane sulphonate, oxalate, propionate, succinate, tartrate, phosphate, glutamate. As non-restrictive examples of base addition salts, salts based on the following are mentioned: alkali metals and alkaline earth metals (lithium, potassium, sodium, calcium, magnesium, aluminium), quaternary ammonium salts, amine cations (methylamine, ethylamine, diethylamine, etc.).

Esters of the proteins according to the present invention involve all pharmaceutically acceptable esters known by a person skilled in the art. It is within the general knowledge of a skilled person how to form esters using a surface functional group of a protein. These functional groups are typically alcoholic and carboxylic functional groups.

In respect of the present invention prodrugs are compounds that transform in vivo into a protein according to the present invention. Transformation can take place for example in the blood during enzymatic hydrolysis. In the prodrug form the compound is not active: it cannot fulfil its function. For example, if any of the amino acid residues of the inhibitory loop is covalently modified with a bulky compound, the loop cannot efficiently interact with any proteinases including MASP-2. If the chemical modification can be removed by a chemical reaction, e.g. hydrolysis, catalysed by a host enzyme, the prodrug will be transformed to active drug. Protein modifications resulting in prodrugs are known for a person skilled in the art (Tobin 2014, Gou 2016).

The proteins of the general formula (Ih) according to the invention can be used in human pharmaceutical preparations, where one or more additives are needed to reach the appropriate biological effect. Such preparations may be pharmaceutical preparations combined, for example, with matrices ensuring controlled active agent release, widely known by a person skilled in the art. Generally, matrices ensuring controlled active agent release are polymers that, when entering the appropriate tissue (e.g. blood plasma), decompose, for example in the course of enzymatic or acid-base hydrolysis (e.g. polylactide, polyglycolide).

In the human pharmaceutical preparations according to the invention other additives known in the state of the art can also be used, such as diluents, fillers, pH regulators, substances promoting dissolution, colouring additives, antioxidants, preservatives, isotonic agents, etc. These additives are known in the state of the art.

Preferably, the human pharmaceutical preparations according to the invention can be entered in the organism via parenteral (intravenous, intramuscular, subcutaneous, etc.) administration. Taking this into consideration, preferable pharmaceutical compositions may be aqueous or non-aqueous solutions, dispersions, suspensions, emulsions, or solid (e.g. powdered) preparations, which can be transformed into one of the above fluids directly before use. In such fluids suitable vehicles, carriers, diluents or solvents may be, for example water, ethanol, different polyols (e.g. glycerol, propylene glycol, polyethylene glycols and similar substances), carboxymethyl cellulose, different (vegetable) oils, organic esters, and mixtures of all these substances.

The preferable formulations of the pharmaceutical preparations according to the invention include among others infusions, tablets, powders, granules, suppositories, injections, syrups, etc. One of the preferred administration routes of proteins and peptides is the intranasal delivery to bypass the blood-brain barrier (Meredith 2015). Therefore, preferred preparations include intranasal delivery systems, like. e.g. cyclodextrins, inhaled solutions, etc.

The administered dose depends on the type of the given disease, the patient's sex, age, weight, and on the severity of the disease. In the case of oral administration, the preferable daily dose may vary for example between 0.01 mg and 1 g, in the case of parenteral administration (e.g. a preparation administered intravenously) the preferable daily dose may vary for example between 0.001 mg and 1 g in respect of the active agent. A person skilled in the art finds it obvious that the dose to be selected depends very much on the molecular weight of the given protein used.

Furthermore, the pharmaceutical preparations can also be used in liposomes or microcapsules known in the state of the art. The proteins according to the invention can also be entered in the target organism by state-of-the-art means of gene therapy.

One might think that the rest of the protein according to the present invention, i.e. those parts that are not the sequence of general formula (Ih), does not significantly influence selectivity. However, as it is obvious for a person skilled in the art, a given protein may have parts beyond the sequence part according to the invention, which may influence the inhibitory activity shown towards the MASP-2 target enzymes. Practically, such undesired effects should be filtered out by performing experiments, and planning these in advance is quite difficult and uncertain according to the state of the art. Performing said experiments (i.e. measuring MASP-2 inhibitory activity of certain protein constructs) is within the scope of a person skilled in the art, however, it is described below in Example 4.2.

The proteins according to the invention can be preferably used in various kits, which can be used for measuring and/or localising the MASP-2 enzyme. Such use may extend to competitive and non-competitive tests, radioimmunoassays, bioluminescent and chemiluminescent tests, fluorometric tests, enzyme-linked assays (e.g. ELISA), immunocytochemical assays, etc.

In accordance with the invention, kits are especially preferable, which are suitable for the examination of the potential inhibitors of the human MASP-2 enzyme, e.g. in competitive binding assays. With the help of such kits a potential inhibitor's ability of how much it can displace the protein according to the invention from the MASP-2 enzyme can be measured. In order to detect it, the protein according to the present invention needs to be labelled in some way (e.g. incorporating a fluorescent group or radioactive atom, or other labelling means known according to the state of the art).

The kits according to the invention may also contain other solutions, tools and starting substances needed for preparing solutions and reagents, and instruction manuals. Here, under instruction manual a simple reference to an online manual is also understood.

The proteins containing any sequence according to general formula (Ih) can also be used for screening compounds potentially inhibiting the human MASP-2 enzyme. In the course of such a screening procedure a protein containing any sequence according to general formula (Ih) is used in a labelled (fluorescent, radioactive, etc.) form in order to ensure detectability at a later point. The preparation containing such a protein is added to the solution containing the human MASP-2 enzyme, or to a sample containing surface immobilized human MASP-2, respectively, in the course of which the protein binds to the human MASP-2 enzyme. Following the appropriate incubation period, a solution containing the compound/compounds to be tested is added to this preparation, which is followed by another incubation period. The compounds binding to the human MASP-2 enzyme (if the tested compound binds to the surface of the human MASP-2 enzyme partly or completely at the same site where the sequence according to the invention is located, i.e. in a competitive manner, or somewhere else, but its binding alters the conformation of the MASP-2 enzyme in such a way that it loses its ability to bind to the protein, i.e. in a non-competitive manner) displace the labelled protein from the human MASP-2 molecule to the extent of their inhibiting ability. The concentration of the displaced proteins can be determined by using any method suitable for detecting the labelling (e.g. fluorescent or radioactive) used on the protein molecules of the present invention. The incubation periods, washing conditions, detection methods and other parameters can be optimised in a way known by the person skilled in the art. The screening procedure according to the invention can also be used in high-throughput screening (HTS) procedures, as is obvious for a person skilled in the art.

The proteins according to the present invention, i.e. proteins containing the sequence of general formula (Ih), can be used first of all in the medical prevention or treatment of diseases, in the case of which the inhibition of the operation of the complement system has preferable effects. Consequently, the present invention also relates to the use of proteins in the production of medicaments for the prevention or treatment of such diseases. In accordance with this, diseases can be selected preferably from the following non-limiting groups:

(1) ischemia-reperfusion (IR) injuries (especially following recanalyzation after arterial occlusion due to thrombosis or other obstructive diseases), including those occurring after myocardial infarction (e.g. treated by percutaneous coronary interventions or thrombolysis), coronary bypass surgery, organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), rheumatoid arthritis (RA), juvenile idiopathic arthritis, age-related macular degeneration, systemic lupus erythematosus (SLE), atypical hemolytic uremic syndrome (aHUS), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; (3) neurodegenerative diseases, preferably Alzheimer's disease, Huntington's disease and Parkinson's diseases and multiple sclerosis.

The proteins according to the invention can also be used for isolating the human MASP-2 protein, by immobilising proteins of the present invention and making the preparation made in this way come into contact with the solution presumably containing the human MASP-2 enzyme. If this solution really contains the human MASP-2 enzyme, it will be anchored via the immobilised protein. This procedure can be suitable both for analytical and preparative purposes. The solution containing the human MASP-2 enzyme can be a pure protein solution, an extract purified to different extents, tissue preparation, etc.

According to the present invention, by proteins containing the sequences according to the invention we mean the following. By such protein we mean any amino acid sequence, which consists of the sequence according to general formula (Ih) at least. However, preferably this sequence is a part of a larger protein (i.e. host protein) to make sure that the two extreme members of the sequence of general formula (Ih) according to the invention (that is, amino acids marked X₁ and X₅) are situated at an appropriate distance from each other. First of all, this appropriate distance can be ensured by the appropriate molecular environment, that is, by a protein of an appropriate spatial structure. Due to the appropriate distance between these two extreme amino acids, the sequence part according to the invention can assume the appropriate optimal geometry for inhibiting the MASP-2 enzyme. For this reason, the distance measured between the alpha carbon atoms of the two extreme amino acids X₁ and X₅ of the general formula (Ih) is preferably 20±4 Å. A person skilled in the art finds it obvious that the appropriate distance between the two extreme amino acids of the sequence according to the invention can be ensured by inserting it in a larger protein, and also by adding a suitable shorter sequence part, even a few amino acids, and by creating a covalent or ionic bond between them. For example, cysteine side chains can be inserted a few positions before and after the two extreme amino acids mentioned above, and by creating appropriate conditions between these cysteine side chains, a covalent disulfide bridge can be created. It follows from the above that according to the present invention shorter peptides and modified peptides are also regarded as proteins.

According to the present invention the protein according to the invention is preferably a protein within the Kunitz family, carrying the 7-residue sequence according to the general formula (Ih) immediately following the conserved Gly residue at position 12 of SEQ ID NO: 22 (i.e. X12) of the Kunitz domain and therefore occupying the segment from position 13 to position 19 according to the position numbering defined for the Kunitz domains in SEQ ID NO: 22.

According to the invention, especially preferably the host protein is a TFPI-D2 protein within the Kunitz family. The sequence of the TFPI-D2 domain (identified within the sequence of TFPI-1; UniProt ID P10646, as residues 121-178) is the following (SEQ ID NO: 23):

KPDFCFLEEDPGICRGYITRYFYNNQTKQCERFKYGGCLGNMNNFETLEECKNICEDG,

where the underlined part indicates the sequence part to be replaced with the sequence part according to the general formula (Ih).

Conservation rules observed within the Kunitz family are described in Table 14 of the U.S. Pat. No. 5,994,125 patent. These conservation rules were taken into consideration. The most important features are indicated in FIG. 4, which depicts the above sequence (SEQ ID NO: 23) as follows: bold highlights fully conserved residues while italic indicates positions that are represented by only 2-3 amino acid types within the Kunitz family. Positions that were randomized during our work in the TFPI-D2 protease binding loop (i.e. the inhibitory loop) library are underlined in FIG. 4. Only non-conserved residues have been randomized. Note here, that the conserved cysteine in the region to be replaced was not randomized, as it is a conserved residue.

For the better understanding the above concept behind our present invention, as an example, for the sequence of the present invention we take FCRAVKR (SEQ ID NO: 1) as one of the preferred sequences of general formula (Ih) and use the TFPI-D2 domain (SEQ ID NO: 23) as the host protein. Here, the sequence ICRGYIT in SEQ ID NO: 23 is changed to the sequence according to SEQ ID NO: 1, and the resulting sequence is SEQ ID NO: 11. This protein according to SEQ ID NO: 11 is a potent MASP-2 inhibitor of the present invention:

KPDFCFLEEDPGFCRAVKRRYFYNNQTKQCERFKYGGCLGNMNNFETLEE
CKNICEDG,

where the underlined part indicates the sequence part of the general formula (Ih). This sequence (SEQ ID NO: 11) is to be regarded exclusively as a preferred embodiment for demonstrating the invention, and not as the limitation of the invention.

As other preferred embodiments, where the host protein is the TFPI-D2 domain, SEQ ID NO: 12 to SEQ ID NO: 20 can be obtained in a similar way, as obvious for a person skilled in the art.

Obtaining further proteins of the present invention where the host protein is not the TFPI-D2 domain, but is a member of the Kunitz family according to the above detailed definition, can similarly be obtained, as obvious for a person skilled in the art. Here, the segment from position 13 to position 19 according to the position numbering defined for the Kunitz domains in SEQ ID NO: 22 shall be replaced by the sequence according to the general formula (Ih).

Obtaining further proteins of the present invention where the host protein is not the member of the Kunitz family, can similarly be obtained, as obvious for a person skilled in the art. In this case, the skilled person will identify a surface loop or the like in the potential host protein, where about seven residues will be substituted to the sequence of the general formula (Ih). In this respect those proteins fall also within the scope of the present invention the host of which are member of the Kunitz family, however, partly or fully another part (i.e. not the segment of from position 13 to position 19 in SEQ ID NO: 22) of the host protein is substituted to the sequence of the general formula (Ih).

### Phage Display

The proteins according to the invention were developed using the phage display method described below.

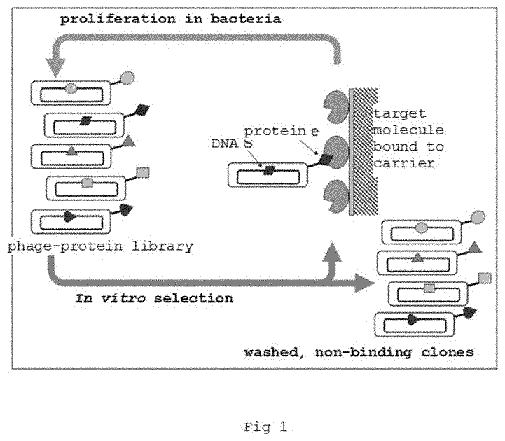

The phage display is suitable for the realisation of directed in vitro evolution of proteins and peptides. The main steps of the state-of-the-art procedure (Smith 1985) is depicted in FIG. 1. In the course of this procedure the gene of the protein involved in evolution is linked to a bacteriophage envelope protein gene. If phage or bacteriophage is mentioned throughout the present description, class I filamentous phage e.g. M13 bacteriophage is meant. In this way, when the bacteriophage is created, a fusion protein is produced which becomes incorporated into the surface of the phage. The phage particle carries the gene of the foreign protein inside, while on its surface it displays the foreign protein. The protein and its gene are physically linked via the phage. For directed protein evolution, we change the codons of the gene coding it, carefully determined by us. Numerous codons can be changed at the same time using combinatorial mutagenesis based on a mixture of synthetic oligonucleotides. The position of the mutations and variability per position is determined at the same time.

After creating a DNA library containing typically several billions of variants and entering it into bacteria, the phage protein library is created. Each phage displays only one type of protein variant and carries only the gene of this variant. The individual variants can be separated from each other using analogue methods to affinity chromatography, on the basis of their ability to bind to a given target molecule chosen by the researcher. Generally, the target molecule is linked or bound to a surface and serves as the stationary phase of the affinity chromatography process. At the same time, as opposed to simple protein affinity chromatography, the so-called protein-phages that were selected in this way and carry target-binding variants of the displayed protein have two important characteristic features. On the one part, they are able to multiply in E. coli cells, on the other part these particles also display the selected variants of the displayed protein and carry the coding genes wrapped in the phage particles.

During evolution, instead of examining individual mutants, in actual fact billions of experiments are performed simultaneously. Binding variants are multiplied, and after several cycles of selection-multiplication a population rich in functional variants is obtained. From this population, individual phage clones displaying one selected variant of evolved protein are examined in functional tests. The phage protein variants found appropriate during the tests are identified by sequencing the physically linked gene. Besides the individual measurements, through the sequence analysis of an appropriately large number of function-selected clones it is also revealed what amino acid sequences enable fulfilling the function. In this way, a database based on real experiments is prepared which makes it possible to elaborate a sequence-function algorithm. The variants found the best on this basis are also produced as independent proteins, and these are examined in more accurate further tests.

We ourselves—i.e. the inventors of the present invention—developed the vectors suitable for phage display from the vectors available in commercial distribution, they will be described later.

### Selecting the Inhibitor Scaffold

When selecting the inhibitor scaffold, it was a condition that the marked inhibitor structure should be: canonical, highly efficient on human trypsin and small. If a peptide or protein is inhibiting trypsin, it means that its loop is resistant to the tryptic cleavage or despite such a cleavage it maintains its activity, and furthermore such a peptide or protein will probably fit to the active center of a trypsin-like protease. A further expectation in connection with the new inhibitor scaffold was that it should have an inhibitory loop with the least possible structural constraints, as this loop will be evolved to the inhibitory loop of the proteins of the present invention. Finally, the scaffold was decided to have human origin in order to minimize the risk of immunogenicity of the evolved MASP-2 inhibitor when used in targeting human diseases. The structure of the member named TFPI-D2 (which, as already mentioned, equals the second domain (residues 121-178) of the human Tissue Factor Pathway Inhibitor-1 protein (TFPI-1; UniProt ID P10646)) in the Kunitz inhibitor family fulfils these requirements: it is canonical, it can inhibit several different types of human serine protease (e.g. trypsin), and with its length of 58 amino acids it can still be regarded small. As compared to the SFTI inhibitor scaffold disclosed in international patent application Pub. No. WO 2010/136831, as well as to the SGCI inhibitor scaffold disclosed in international patent application Pub. No. WO 2012/007777 there are several positions in the inhibitory loop that harbour residues having side chains, which do not participate in creating the internal structure of the molecule, but they interact with the target protease. It seemed justified that the guided evolutionary modification of positions in the inhibitory loop that have no known internal structure stabilizing roles (e.g. are not cysteines participating in a disulphide bridge) would result in more selective inhibitors of a higher affinity with the added benefit of having a human origin scaffold.

### Creating a Library

As the basic molecule of the library we chose the TFPI-D2 protein domain, which is a potent inhibitor of factor Xa, a negligibly weak inhibitor of MASP-2 (see below), and is a serine-protease inhibitor within the so-called Kunitz family (family 12 of the MEROPS Peptidase Database at http://merops.sanger.ac.uk/inhibitors/). The parent molecule of TFPI-D2, Tissue Factor Pathway Inhibitor 1 (TFPI-1) is a natural, low abundance inhibitor of blood coagulation in the serum (its total serum concentration is about 2.5 nM but only about 10% of TFPI-1 circulates freely) that does not inhibit the complement system. It contains three consecutive Kunitz-type domains. The first two domains inhibit factor VIIa and factor Xa, respectively. It was also shown that the second Kunitz-type domain of TFPI-1 (TFPI-D2) inhibits MASP-2 (Wouters et al., 2011 [conference poster]; Keizer et al., 2015). Since this inhibitory effect is extremely weak (IC50=10 μM in solid phase ELISA assay with diluted serum) especially compared to the plasma concentration of TFPI-1, the physiological relevance of this interaction should be negligible. On the other hand, this very weak interaction of TFPI-D2 with MASP-2 indicated that TFPI-D2 might be evolved to a potent MASP-2 inhibitor.

The typical inhibitors (inhibitor domains) within the Kunitz family consist of about 60 amino acid residues, contain a twisted two-stranded antiparallel beta sheet followed by a C-terminal alpha helix, and their fold is stabilized by three disulfide bridges in the following arrangement:

CₐX₈C_{b}X₁₅C_{c}X₇C_{b}X₁₂C_{c}X₃Cₐ

In other words, the six cysteines form disulfides described by the abcbca pattern. The canonical protease inhibitory loop is located on the peptide segment immediately preceding the first beta-chain of the beta-sheet. Based on the Kunitz domain definition described above in detail, said loop covers the X12-X20 positions of SEQ ID NO: 22. This segment is anchored to and thereby stabilized by another peptide loop segment, which runs behind it in an antiparallel orientation.

When creating the library, our aim was the complete randomisation of the protease inhibitory loop (except the conserved X12 glycine and X14 cysteine, see below and FIG. 4). Generally, the loop is determined with positions P4-P4′ (in the case of substrates the environment of the cleaved bond within +/−4 amino acids; nomenclature: Schechter & Berger 1967). The highly conserved X12 glycine at the P4 position and the highly conserved X14 cysteine at the P2 position situated in the P4-P4′ region, have key importance in forming and stabilizing the canonical conformation of the inhibitory loop and therefore were left intact. In the other six positions, we allowed the presence of all twenty amino acids.

The initial TFPI-D2 inhibitor loop's amino acid sequence: GICRGYIT (residues from position 12 in the SEQ ID NO: 23).

The amino acid sequence of the inhibitory loop characteristic of the library is GxCxxxxx (residues from position 12 in the SEQ ID NO: 22),

where x can be any one of the twenty amino acids during the evolution process. The underlined part indicates the so-called P1 group, which generally bears outstanding significance from the aspect of specificity, as it reaches into a deep binding pocket of the enzyme responsible for primary selectivity.

In order to be able to select high-affinity binding molecules during phage display, it is essential that the binding molecule displayed should be presented in a low copy number per phage, ideally in one single copy (monovalent phage display). By this, seemingly high-affinity binding deriving from simultaneous binding to several anchored target molecules, i.e. avidity can be avoided. In the system used by us, the phage-TFPI-D2 library was created through a glycine-serine linker as the N-terminal fusion of the p8 main envelope protein. We have previously showed that the 35-aminoacid, three disulphide containing Pacifastin family SGCI molecule displayed on M13 phage analogously as P8 fusion protein appears on the phage surface in one single copy (Szenthe 2007). We expected a similar monovalent display with our TFPI-D2 system.

Before the N-terminus of the TFPI-D2 library members, we also inserted a linear epitope tag recognisable by monoclonal antibodies, using an appropriate distance-keeping peptide linker. This was the so-called “Flag-tag”, which served the purpose of demonstrating successful display of any library member on phage, even those that do not bind to the target proteinase, MASP-2 (or any other given proteinase).

### EXAMPLES

Below the present invention is described in detail on the basis of examples, which, however, should not be regarded as examples to which the invention is restricted.

Through the examples we show how the phage library was created (Example 1), describe phage selection (Example 2) and introduce the results (Example 3). In Example 4 the method of the heterologous expression of the inhibitors is described together with the relating analytical studies.

### Example 1: Creating the Phage Library

### 1.1. Constructing the Phage Display Vector

The vector constructed for displaying TFPI-D2 was based on the pSFMI-pro-lib vector (Kocsis 2010), which had been created from the commercial vectors pBluescript II

KS(−) (Stratagene), pMal-p2X (NEB), and M13KO7 helper phage. The pSFMI-pro-lib vector was intensely modified in several consecutive steps. The SGCI coding region was removed, a unique XhoI site was replaced with a unique HindIII site; the original unique KpnI (Acc65I) site and one of the two SacI sites was deleted, and a new unique Kpn2I (BspEI) as well as a new unique EcoRI site was introduced. The final product was able to accommodate the coding DNA of TFPI-D2 such, that the protein became flanked by Ser/Gly linkers on both termini and displayed as a p8 coat protein fusion on the surface of M13. Moreover, the construct also provided the displayed protein with an N-terminal FLAG-tag for easy assessment of display efficiency.

We designed a codon optimized gene for the 2nd domain of the Tissue Factor Pathway Inhibitor-1 protein, supplied it with the necessary flanking regions for cloning into the display vector between Kpn2I (BspEI) and SacI sites, and obtained the synthetic DNA construct from Life Technologies. The synthetic gene had the following sequence (SEQ ID NO: 24):

TCCGGAGGCTCGGGCAAACCGGACTTCTGCTTCCTGGAAGAAGACCCGGG
TATCTGCCGTGGTTACATCACCCGTTACTTCTACAACAACCAGACCAAAC
AGTGCGAACGTTTCAAATACGGTGGTTGCCTGGGTAACATGAACAACTTC
GAAACCCTGGAAGAATGCAAAAACATCTGCGAAGACGGTGGCGGCAGCGG
CGGCAGCGGCGGGAGCTC

The flanking SerGly linker coding regions are from position 1 to 16 and from position 190 to 218, while the Kpn2I and SacI restriction sites are from position 1 to 6 and from position 213 to 218.

Subcloning the synthetic DNA into the display vector resulted in the pTFPI-D2-pro-lib vector.

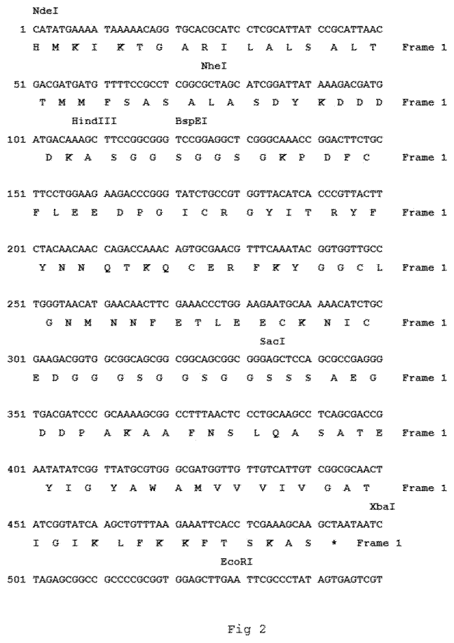

FIG. 2 depicts the DNA and amino acid sequence of the fusion gene made to display TFPI-D2 on the surface of the M13 bacteriophage.

The different functional parts of the fusion protein as follows (numbering is according to nucleotides in FIG. 2.): from position 4 to 81 is the malE periplasmic signal sequence, from position 85 to 108 is the FLAG-tag, from position 111 to 135 is a first Ser-Gly linker, from position 136 to 309 is the TFPI-D2 domain, from position 310 to 342 is a second Ser-Gly linker, and from position 343 to 492 is the major coat protein (p8) of the M13 phage. The restriction endonuclease cleavage sites used during the construction of the fusion gene are shown in FIG. 2 above the DNA sequence of their cleavage sites. Residues that are targeted by directed evolution are shown from nucleotide position 172 to 174 and from 178 to 192.

Display efficiency of TFPI-D2 on the surface of M13 phage was tested in phage ELISA assays. Bovine trypsin (Sigma-T1426, 20 μg/ml, 100 μl/well) and αFLAG-tag mAb (Sigma-F3165, 2000-fold dilution, 100 μl/well) diluted in 200 mM Sodium-carbonate pH 9.4 (coating buffer) were immobilized on Nunc MaxiSorp 96-well ELISA plates (442404) for 2 hours at room temperature. The plastic surface was blocked with 200 μl/well blocking solution (5 mg/ml bovine serum albumin dissolved in phosphate buffered saline pH 7.4 (PBS)). After one hour of blocking at room temperature the plate was washed with PBS supplemented with 0.05% Tween-20 (wash buffer). Separate wells were only blocked with 200 μl/well blocking solution and served as negative control. Concentration of a stock solution of TFPI-D2 displaying phages (isolated as described later in 1.3.1.1. and 1.3.1.2. using E. coli XL1 Blue cells) dissolved in PBS was estimated by measuring the optical density (absorbance) of the solution at 268 nm (O.D._{268 nm}) where O.D._{268 nm}=1.0 corresponds to ˜5×10¹² phage/ml. An aliquot of the phage stock was diluted into blocking solution supplemented with 0.05% Tween-20 (PBT buffer, the name referring to the components PBS, BSA and Tween-20) to have a final concentration of 5×10¹² phage/ml. Then, 3-fold serial dilution of phages was made in PBT and 100 μl/well aliquots of the serially diluted samples were transferred to the three ELISA surfaces mentioned above, i.e. one coated with trypsin and blocked with BSA, the other coated with

αFLAG-tag mAb and blocked with BSA and the third one not coated but blocked with BSA. The last one served as a control to assess non-specific binding. After 2 hours of incubation the plate was washed and 100 μl/well horseradish peroxidase(HRP) conjugated αM13 mAb (GE Healthcare Life Sciences—27-9421-01) diluted 5000-fold in PBT buffer was transferred to the wells. After 30 minutes of incubation the plate was washed and 100 μl/well TMB peroxidase substrate (3,3′,5,5′-Tetramethylbenzidine, Pierce, cat #34028) was added to the wells. After the signal developed the reaction was stopped by adding 50 μl/well 1 M HCl and the plate was read at 450 nm on an Amersham Biosciences BioTrak II microplate reader. The signals on trypsin coated and αFLAG-tag mAb coated wells were corrected with the signals produced by the same amount of phage on BSA coated control surfaces.

### 1.2. Preparation of a Kunkel-Template

In the first step, from pTFPI-D2-pro-lib phagemid a single-stranded Kunkel-template was prepared, in which stop codons were inserted using Kunkel's method (Kunkel 1985) in the positions to be randomised at a later point. The role of the stop codons is to eliminate possible wild-type TFPI-D2 backgrounds while creating the DNA library. The mutagenesis used when creating the library is never 100% efficient, some of the created population is of the same sequence as the template. In our case this population does not appear as a displayed peptide, as it contains numerous stop codons.

The DNA library was also created using Kunkel's method. For this we used degenerate oligonucleotides. The DNA library created in this way was introduced into supercompetent cells by electroporation. The phage protein library was created by the helper phage infection of the cell culture.

### 1.3. Creating the DNA Library

### 1.3.1. Creating Stop Mutant Phagemid by Kunkel Mutagenesis

1.3.1.1. Transformation of CJ 236 E. coli Strain
1 μl (˜100 ng) pTFPI-D2-pro-lib phagemid

### 4 μl 5×KCM (0.5 M KCl, 0.15 M CaCl2, 0.25 M MgCl₂)

15 μl distilled water
The DNA solution was cooled on ice.
We added 20 μl CJ236 cells (NEB) and the sample was incubated for 20 minutes on ice. Then we left the cells alone for 10 minutes at room temperature, and after adding 200 μl LB medium we shook it for 30 minutes at 37° C. The cells were spread onto an LB-agar+ampicillin (100 μg/ml) plate and grown overnight at 37° C.

### 1.3.1.2. Production and Isolation of Uracil-Containing Phage

From a separate colony, cells were inoculated in 2 ml 2YT/ampicillin (100 μg/ml), chloramphenicol (5 μg/ml) medium and grown overnight, shaken at 37° C. On the following day 30 μl culture was inoculated in 3 ml medium of the same composition. As soon as the light dispersion of the cell suspension measured at 600 nm (O.D.₆₀₀ nm) reached 0.4, it was infected with M13-K07 helper phage (NEB) allowing at least 10 phages per coli cell on average. After shaking it for 30 minutes at 37° C. the cells were added to 30 ml 2YT/ampicillin (100 μg/ml), kanamycin (25 μg/ml) medium. The cells were shaken for 16 more hours at 37° C. Then the cells were isolated from the culture by centrifugation (10,000 rpm, 10 minutes, 4° C.), and from the supernatant containing the phages. The phages were precipitated in a clean centrifuge tube by adding ⅕ volume PEG/NaCl solution (20% PEG 8000, 2.5 M NaCl). After thoroughly mixing in the precipitation agent, the sample was left alone for 20 minutes at room temperature. Then the phage particles were settled by centrifuging (12,000 rpm, 10 minutes, 4° C.). After pouring off the supernatant carefully and putting back the tube in the same position, the liquid stuck to the wall of the tube was collected by centrifuging it for a while (1,000 rpm, 1 minute, 4° C.) and then it was removed with a pipette. The phages were suspended in 800 μl PBS, and the remaining cell fragments were removed from the sample by centrifuging it in a microcentrifuge (12,000 rpm, 10 minutes) and transferring the supernatant into a clean microcentrifuge tube. The supernatant obtained in this way contained pure phages.
1.3.1.3. Isolation of Single-Stranded DNA from Phages
From the nearly 800 μl phage, with the help of a QIAgen Spin M13 kit (cat. no. 27704), single-stranded DNA (ssDNA) was isolated following the manufacturer's instructions. The amount of the pure ssDNA was determined on the basis of UV light absorption at 260 nm.

### 1.3.1.4. Kunkel Mutagenesis

Stop mutations were introduced with the TFPI D2 P3-P4′ STOP oligonucleotide (SEQ ID NO: 25):

5′-CCTGGAAGAAGACCCGGGTTAATGCTAATAATAATAATAACGTTACT
TCTACAACAACCAGACC-3′

The TAA stop codons are from position 20 to 22 and from position 26 to 40

### 1.3.1.4.1. Oligo Phosphorylation

2 μl mutagenesis oligo (330 ng/l)
2 μl 10×TM buffer (0.5 M Tris-HCl pH 7.5, 0.1 M MgCl₂)

### 2 μl 10 mM ATP

### 1 μl 100 mM DTT

12 μl distilled water
1 μl polynucleotide kinase (NEB, 10 U/μl)
The reaction was incubated for 30 minutes at 37° C.

### 1.3.1.4.2. Oligo-Template Annealing

1 μg ssDNA
2 μl from the kinase oligo reaction mixture
2.5 μl 10×TM buffer
Distilled water up to a final volume of 25 μl
Incubation: 90° C. 1 minutes, 50° C. 3 minutes, then after centrifuging it for a while it was put on ice.

### 1.3.1.4.3. Polymerisation and Ligation

The following were added to the above DNA solution:

### 1 μl 10 mM ATP

1 μl 25 mM dNTP

### 1.5 μl 100 mM DTT

0.6 μl T4 ligase (NEB, 400 U/μl)
0.6 μl T7 polymerase (NEB, 10 U/μl)

The reaction was incubated for 2 hours at 37° C.

The whole mixture was run on 1% agarose gel and the product of the desired size was cut out from the gel. From this piece of gel, with the help of a QIAgen Gel Extraction kit (cat. no. 28706), the Kunkel product was isolated in 30 μl elution buffer (EB). With the Kunkel product XL1 Blue cells were transformed as described in 1.3.1.1. using 10 μl DNA. From individual colonies cell cultures were grown in LB/ampicillin (100 μg/ml) medium. From the cells, the phagemid was isolated with a Fermentas GeneJet plasmid miniprep kit (# K0502), following the manufacturer's instructions. The DNA construction was checked via sequencing with Big Dye Terminator v3.1 cycle Sequencing Kit (Applied Biosystems; cat #4336917) system for the sequencing PCR reaction. The product of the sequencing reaction was run by BIOMI Kft. (Gödöllő). The name of the vector created in this way: pTFPI-D2-p8-STOP phagemid.

### 1.3.2. Library Kunkel Mutagenesis

The library mutagenesis was realised in a similar way as described above in point 1.3.1.4., but using ten times the amounts determined therein. The library oligo is analogous with the stop mutation oligo, but in this case, there are degenerate NNK triplets (using the IUPAC coding relating to degenerate oligonucleotides) in the place of the TAA stop codons. The sequence of the TFPI D2 P3-P4′ library primer was the following (SEQ ID NO: 26):

5′-CCTGGAAGAAGACCCGGGTNNKTGCNNKNNKNNKNNKNNKCGTTACT
TCTACAACAACCAGACC-3′

The degenerate NNK codons are from position 20 to 22 and from position 26 to 40.

Oligo phosphorylation was performed as described above in 1.3.1.4.1. To create the library ten times the amount of the oligo was used in oligo-template annealing, so all the oligo created during the kinase reaction was used. The template for the mutagenesis was the uracil-containing ssDNA carrying the stop codons, which was created from the pTFPI-D2-p8-STOP phagemid obtained as a result of the procedure described above in detail, in CJ236 cells, by M13K07 helper phage infection. For creating the library ten times the amount of the template was used: 20 μg and the volume of the annealing reaction was also increased by ten times to 250 μl. The incubation periods were extended: 90° C. 2 minutes, 50° C. 5 minutes. The polymerization and ligation reaction was incubated for 3 hours at 37° C.

The product was purified with Qiagen Gel Extraction kit. It was not isolated from gel, only purified using two columns. Elution took place in 2×30 μl USP distilled water.

### 1.4. Electroporation, Multiplication of the Phage Library

The library was introduced to the supercompetent cells via electroporation. Our aim was to introduce the plasmid to as many cells as possible, so that our library contained 10⁸-10⁹ pieces.

The DNA library, which was situated in USP distilled water so it was salt-free, was added to 2×350 μl supercompetent cells. 30 μl of library DNA was electroporated into 350 μl of supercompetent cells and the process was repeated with the other half of the DNA library. The operation was performed in a cuvette with a gap size of 0.2 cm, according to the following protocol: 2.5 kV, 200 Ohm, 25 μF.

After electroporation, the cells were carefully transferred into 2×25 ml of SOC medium, incubated for 30 minutes by shaking at 200 rpm, at 37° C., then a 10 μl sample was taken, a tenfold, 8-member serial dilution was made from it and 10 μl from each dilution was dripped onto [LB], [LB; 100 μg/ml ampicillin] and [LB; 10 μg/ml tetracycline] plates, and it was grown overnight at 37° C. After taking the above sample, the rest of the 2×25 ml culture was infected with 2×250 μl M13KO7 helper phage (1×10¹³ PFU/ml), shaken at 37° C. for 30 minutes at 220 rpm, and then the whole product was inoculated into 2×500 ml [2YT; 100 μg/ml ampicillin; 25 μg/ml kanamycin] medium. The culture was grown in two 2-litre baffled Erlenmeyer flasks at 37° C., at 220 rpm, for 18 hours.

On the basis of titration our library contained 5×10⁸ variants.

### Example 2: Selection of the Library on the Human MASP-2 Enzyme and Independently, in Parallel, on the Rat MASP-2 Enzyme

### 2.1. The Target Enzymes

The MASP-2 targets consist of a serine-protease (SP) domain and two complement control protein domains (CCP-1,-2) (Gál 2007). These are recombinant fragment products, which carry the catalytic activity of the entire molecule (‘catalytic fragment’). The proteins were produced in the form of inclusion bodies, from which the conformation with biological activity was obtained by renaturation. Purification was performed by anion and cation exchange separation. The activity of the proteins was tested in a solution and also in a form linked to the ELISA plate. (For the amino acid sequence and the precise details of production of the human MASP-2 target see Ambrus 2003). The rat MASP-2 target was produced similarly to the human target. The catalytic fragment of rat MASP-2 starts with Gln298 and ends with Phe685 according to UniProt numbering (entry Q9JJS8). Cloning was carried out as in the case of human MASP-2 described in Ambrus 2003. As a result of cloning, the recombinant protein was produced with an extra Met-Thr dipeptide segment at the N terminus. The rat recombinant protein was expressed, refolded and purified following the procedure used earlier at the human protein fragment. The amino acid sequence of the catalytic fragment of rat MASP-2 (MASP2cf) used in the library selection is SEQ ID NO: 27.

The data of the targets used during selection are the following: human MASP-2cf CCP1-CCP2-SP: Mw=44017 Da, c_{stock}=0.13 g/l rat MASP-2cf CCP1-CCP2-SP: Mw=42309 Da, c_{stock}=0.35 g/l

### 2.2. Steps of Selection

### 2.2.1. Isolating the Phages

At the end of the operation described in chapter 1.3, phages were produced in 2×500 ml of culture for 18 hours. In the first step of the selection they were isolated to enable the use of the library immediately for selection.

The cell culture was centrifuged at 8,000 rpm for 10 minutes, at 4° C. The supernatant, which contained bacteriophages, was poured into clean centrifuge tubes, and a precipitating agent ⅕^{th} of its volume was added to it [2.5 M NaCl; 20% PEG-8000]. Precipitation took place at room temperature, for 20 minutes. Then it was centrifuged again at 10,000 rpm for 10 minutes, at 4° C. The supernatant was discarded, it was centrifuged again for a short time, and the remaining liquid was pipetted off. The white phage precipitate was solubilised in 25 ml [PBS; 5 mg/ml BSA; 0.05% Tween-20] buffer. In order to remove residual cell debris, it was centrifuged again at 12,000 rpm for 10 minutes and the supernatant was transferred into clean tubes.

### 2.2.2. The First Selection Cycle

a) Immobilisation: The target molecules were immobilised in separate wells on a 96-well Nunc Maxisorp ELISA plate (cat #442404). During immobilisation, the concentration of human
   MASP-2cf and rat MASP-2cf was 20 μg/ml. Both proteins were diluted in the immobilisation buffer [200 mM Na₂CO₃; pH 9.4], and 100 μl was put in the wells. Both human and rat MASP-2cf were incubated overnight at 4° C. In the first selection cycle 12 wells per target protein were used.
   Every second row was left empty. As negative control only immobilising buffer was put in one row. This row was then treated the same way as the ones covered with target protein.
b) Blocking: The immobilising solution was removed, and 200 μl/well of blocking buffer [PBS; 5 mg/ml BSA] was put onto the plate. It was incubated at room temperature while mixing at 150 rev/min for at least 1 hour.
c) Washing: The ELISA-plate was washed 4 times using 1 l of wash buffer [PBS; 0.05% Tween-20].
d) Selection: The phages of the library isolated as described above were pipetted onto the plate, 100 μl in each well. It was incubated at room temperature while mixing at 110 rev/min, for 2.5 hours.
e) E. coli XL1 Blue culture (created by the inventors of the present invention, publication in press): During the term of the selection, XLI Blue cells were inoculated from a plate freshly picked in advance using an inoculating loop, into 2×30 ml of medium [2YT; 10 μg/ml tetracycline].
   These cells were to be infected with phages eluted from the target proteins. At the time of infection, the cells must be in the phase of exponential growth. A culture with O.D._{600 nm}˜0.3-0.5 was needed, which was obtained by growing it at 37° C., at 220 rpm, for 2-3 hours.
f) Washing: The ELISA-plate was washed 12 times using 3 litres of wash buffer.
g) Elution: Elution was performed using 100 mM HCl solution, 100 μl/well. The acid was applied, shaken for 5 minutes, and then it was drawn from each well one by one. The phages eluted from the individual target proteins were collected in a tube, in which 12×15 μl 1 M Tris-base buffer had been put in advance to quickly neutralise the acid solution containing the phages. The tubes were immediately mixed and placed on ice.
h) Infection: 4.5 ml of XL1 Blue culture in the phase of exponential growth was put in test tubes, and it was infected with 500 μl of phage solution eluted from the target protein. A total number of three infections were performed, with phages eluted from human MASP-2, from rat MASP-2 and from the negative control substance. The cultures were incubated at 37° C., at 220 rpm, for 30 minutes.
i) Titration: A 20 μl sample was taken from each infected culture, it was diluted to 10 times its volume with 2YT medium, and a sequence was prepared with further 10× dilutions. From each point 10 μl was dripped onto a plate [LB agar; 100 μg/ml ampicillin] and grown overnight at 37° C.
j) Infection with helper phage: Directly after sampling, 50 μl M13K07 (1×10¹³ PFU/ml) helper phage was added to each culture in the test tubes, and they were incubated for a further 30 minutes.
k) All infected cultures were transferred into 200 ml medium [2YT; 100 μg/ml ampicillin; 30 μg/ml kanamycin] and incubated at 37° C. while mixing at 220 rpm, for 18 hours.
   The control substance was not treated any further as it was only needed for titration.
l) Enrichment: On the following morning titration was checked. The number of phages eluted from the human MASP-2 was 1.5-2-fold higher than that of phages eluted from the background, while in the case of rat MASP-2 this ratio was 1.

### 2.2.3. The Second Selection Cycle

In this cycle, the same steps were repeated as in the case of the first selection cycle. In this step, each target protein had its own control substance (12 wells), and the phages eluted and multiplied in the previous cycle were placed both on the target and the control protein.

The phages produced for 18 hours were isolated as described above, but at the end they were solubilised in 10 ml of sterile PBT buffer. After the second selection cycle 2.7 ml of fresh exponentially growing XL1 Blue cells was infected with 300 μl of eluted phage. Titration was performed in all four cases (2 target proteins+2 control substances), and then the cultures also infected with helper phage were transferred into 30 ml [2YT; 100 μg/ml ampicillin; 30 μg/ml kanamycin] medium.

After the second selection cycle we determined the number of clones eluted from the specific target coated, BSA blocked wells and divided this number with the number of clones eluted from the target-free, BSA blocked wells. This ratio is referred to as the enrichment values. We obtained an enrichment value of 15-20 for human MASP-2, and a value of 4-5 for rat MASP-2.

### 2.2.4. The Third Selection Cycle

Everything was performed in the same way as in the first and second cycles. After isolation, the phages were solubilised in 2.8 ml of sterile PBS.

After the third selection cycle enrichment values were 500-1000 on human MASP-2, and 80-100 on rat MASP-2.

### 2.3. Testing Individual Clones Using Phage ELISA Assay

In the ELISA test, we were looking for phage clones that are able to bind strongly to their own target protein, while they display significantly lower signals on the BSA coated control surface.
a) Infection: In the case of both the human MASP-2 and the rat MASP-2 10 μl of eluted phage from selection cycle 3 was added to 250 μl of XL1 Blue culture in exponential phase. The eluted phages were diluted previously to contain 200-400 phages/10 μl in order to ensure the high excess of cells during infection. The infected cells were incubated for 30 minutes at 37° C. while mixing the suspension at 220 rpm. Then the cells were spread onto [LB; 100 μg/ml ampicillin] plates, and they were grown overnight at 37° C.
b) Inoculation: individual colonies were inoculated into so-called “single loose” tubes (National Scientific Supply—TN0933-C01) containing 500 μl of medium [2YT; 100 μg/ml ampicillin; 10¹⁰ phage/ml M13KO7 helper phage]. These tubes were arranged similarly to a 96-well ELISA-plate arrangement, they can rotate individually, so in a plate incubator, at 37° C., while mixing at 300 rev/min, they are intensely aerated and are suitable for producing small-volume cultures.
c) Immobilisation: both the human MASP-2 and the rat MASP-2 proteins were immobilised at a concentration of 20 μg/ml in 50 μl/well volume, as described above in connection with selection, on Nunc ELISA Maxisorp plates. Each clone was tested on its own target protein and on BSA coated control surface.
d) After 18 hours, the “single loose” tubes were centrifuged in a rotor suitable for accommodating ELISA plates at 2,500 rpm, for 10 minutes, at 4° C., the supernatant was pipetted into clean tubes. After testing aliquots taken from each tube on phage-ELISA the remaining supernatant, in the interest of killing off the contaminating coli bacteria was heated for 2 hours at 65° C. After this, the samples were stored at −20° C., until used for sequencing.
e) Blocking: The liquid was removed from the immobilised samples, and 200 μl/well of [PBS; 5 mg/ml BSA] blocking buffer was placed in each well. Incubation took place at room temperature, for at least 1 hour, while mixing at 150 rev/min.
f) Washing: The plate was washed 4 times using 1 litre of wash buffer.
g) Phage application: 50-50 μl of the phages produced and isolated as described above were placed in the wells. From the same clone samples were pipetted into a total of 2 wells. Incubation was performed at room temperature, for 1 hour while mixing at 110 rev/min.
h) Washing: The plate was washed 6 times using 1.5 litres of wash buffer.
i) Anti-M13 antibody: 50 μl of monoclonal anti-M13 HRP conjugated antibody (Amersham, cat #27-9421-01) 5,000 times diluted in buffer [PBS; 5 mg/ml BSA; 0.05% Tween-20] was placed in the wells, and then it was incubated for 30 minutes at room temperature while mixing at 110 rev/min.
j) Washing: The plate was washed 6 times with 1.5 litres of wash buffer, and then twice with PBS.
k) Signal development: 50 μl of 1-Step Ultra TMB-ELISA substrate (Pierce, cat #34028) was placed in each well, shaken for a while, and then the reaction was stopped by adding 50 μl of 1M HCl in each well.
l) Reading: absorbance was measured at 450 nm, using BioTrak II (Amersham) plate reading photometer.

We took a sample from phage supernatants in the case of which the intensity of the background was low and which displayed at least three times more intensive signals on their own target protein, and prepared the samples for DNA sequencing. We used 2 μl of supernatant and used the Big Dye Terminator v3.1 cycle Sequencing Kit (Applied Biosystems; cat #4336917) system for the sequencing PCR reaction. It was run by BIOMI Kft. (Gödöllő).

### Example 3: Results

In this example, we describe the results of the tests described in examples 1-2, that is the sequences obtained.

From the phages eluted from human MASP-2 we tested 62 clones using ELISA, and finally we found 43 individual sequences. In the case of rat MASP-2 we obtained 53 individual sequences from 56 ELISA-positive clones.

When interpreting the results, we had to take into consideration that the NNK codon pattern used when constructing the DNA library does not ensure the same initial frequency for the individual amino acids. In the NNK codon pattern an amino acid may have one, two or three codons. Therefore, we performed codon normalisation by dividing all amino acid frequencies by the number of codons the given amino acid is represented by in the NNK set.

After data normalisation, we made sequence logo diagrams about the sequences with the help of WebLogo accessible on the internet (http://weblogo.berkeley.edu/logo.cgi).

We examined which were the preferred amino acids in the individual positions and how much they differed from each other depending on whether they derived from human MASP-2 or rat MASP-2.

The results are shown in the form of two diagrams:

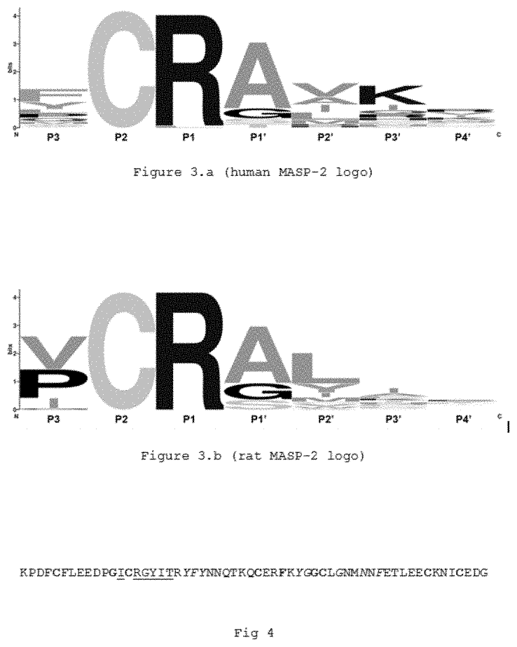

a) the normalised sequence diagram of the clones selected on the human MASP-2 enzyme (FIG. 3.a); and
b) the normalised sequence diagram of the clones selected on the rat MASP-2 enzyme (FIG. 3.b).

As the diagrams show the selected amino acids proportional with their codon normalised frequency, infrequent types are shown in small characters. Therefore, the content of each column in the diagram is shown in the below tables Table 1 and Table 2.

TABLE 1
Content of the codon normalised sequence logo diagram
to the human MASP-2 enzyme

| Position | P3 | P2 | P1 | P1′ | P2′ | P3′ | P4′ |
|---|---|---|---|---|---|---|---|
| Amino acid | F | C(100%) | R | A | V | K | R |
| (frequency) | (33%) | | (98%) | (77%) | (31%) | (45%) | (16%) |
| Amino acid | Y | | L | G | A | I | V |
| (frequency) | (19%) | | (2%) | (12%) | (17%) | (13%) | (16%) |
| Amino acid | L | | | S | I | Q | I |
| (frequency) | (11%) | | | (6%) | (17%) | (9%) | (12%) |
| Amino acid | P | | | T | L | R | K |
| (frequency) | (11%) | | | (5%) | (17% | (9%) | (12%) |
| Amino acid | Q | | | | M | H | M |
| (frequency) | (10%) | | | | (9%) | (6%) | (8%) |
| Amino acid | M | | | | D | S | Q |
| (frequency) | (6%) | | | | (4%) | (5%) | (8%) |
| Amino acid | V | | | | H | F | E |
| (frequency) | (3%) | | | | (4%) | (3%) | (4%) |
| Amino acid | W | | | | S | M | F |
| (frequency) | (3%) | | | | (1%) | (3%) | (4%) |
| Amino acid | A | | | | | N | L |
| (frequency) | (2%) | | | | | (3%) | (4%) |
| Amino acid | T | | | | | L | N |
| (frequency) | (2%) | | | | | (2%) | (4%) |
| Amino acid | | | | | | V | Y |
| (frequency) | | | | | | (2%) | (4%) |
| Amino acid | | | | | | | D |
| (frequency) | | | | | | | (3%) |
| Amino acid | | | | | | | S |
| (frequency) | | | | | | | (3%) |
| Amino acid | | | | | | | H |
| (frequency) | | | | | | | (2%) |

TABLE 2
Content of the codon normalised sequence logo diagram
to the rat MASP-2 enzyme

| Position | P3 | P2 | P1 | P1′ | P2′ | P3′ | P4′ |
|---|---|---|---|---|---|---|---|
| Amino acid | V | C | R | A | L | I | E |
| (frequency) | (45%) | (100%) | (100%) | (68%) | (54%) | (24%) | (16%) |
| Amino acid | P | | | G | Y | A | Y |
| (frequency) | (39%) | | | (20%) | (16%) | (15%) | (16%) |
| Amino acid | I | | | S | I | F | L |
| (frequency) | (14%) | | | (12%) | (8%) | (9%) | (9%) |
| Amino acid | G | | | | M | K | I |
| (frequency) | (2%) | | | | (8%) | (9%) | (8%) |
| Amino acid | | | | | C | T | V |
| (frequency) | | | | | (4%) | (9%) | (7%) |
| Amino acid | | | | | V | N | F |
| (frequency) | | | | | (4%) | (6%) | (5%) |
| Amino acid | | | | | W | Q | M |
| (frequency) | | | | | (4%) | (6%) | (5%) |
| Amino acid | | | | | A | L | N |
| (frequency) | | | | | (2%) | (5%) | (5%) |
| Amino acid | | | | | | S | Q |
| (frequency) | | | | | | (4%) | (5%) |
| Amino acid | | | | | | E | A |
| (frequency) | | | | | | (3%) | (3%) |
| Amino acid | | | | | | R | C |
| (frequency) | | | | | | (3%) | (3%) |
| Amino acid | | | | | | T | G |
| (frequency) | | | | | | (3%) | (3%) |
| Amino acid | | | | | | V | H |
| (frequency) | | | | | | (3%) | (3%) |
| Amino acid | | | | | | G | K |
| (frequency) | | | | | | (1%) | (3%) |
| Amino acid | | | | | | | S |
| (frequency) | | | | | | | (3%) |
| Amino acid | | | | | | | W |
| (frequency) | | | | | | | (3%) |
| Amino acid | | | | | | | R |
| (frequency) | | | | | | | (2%) |
| Amino acid | | | | | | | T |
| (frequency) | | | | | | | (1%) |

The sequence logo diagrams are shown in FIG. 3, where the figure numbers (that is FIGS. 3.a and 3.b) relate to the sequence logo diagram of the above two groups marked a) and b), in the same order.

The sequence logo is the graphic display of the information content and amino acid distribution per position in a set of multiple aligned sequences, using the single-letter abbreviations of the amino acids. In each position, the column height of the logo indicates how even the occurrence of the elements (20 different types of amino acids in our case) is. The less even this occurrence is, the higher the column. In the case of completely even distribution (all 20 amino acids occur in a proportion of 5%) the height is zero. The maximum value belongs to the case, where only one type of element (amino acid) occurs. Within the column the individual amino acids are arranged on the basis of the frequency of occurrence, the most frequent one is at the top. The height of the letter indicating the amino acid is in proportion with its relative frequency of occurrence in the given position (for example, in the case of 50% frequency of occurrence, it is half the height of the column). In the case of colour diagrams, generally amino acids with similar chemical characteristics are shown in the same or in a similar colour, for which we used different shades of grey in the figure belonging to the present patent description.

On the horizontal axis of the sequence logo diagrams the number of the individual positions of the randomised region can be seen, site P1 corresponds to position 3. On the vertical axis, the information content of the positions is determined in bits.

The logo diagrams illustrate the selection taking place in the individual positions. In the initial inhibitor population, the necessity to bind to the MASP-2 enzymes resulted in intensive selection, which especially affected positions 1, 3, 4 and to a lesser extent position 5 (which correspond to P3, P1, P1′ and P2′, respectively).

Human and rat MASP-2 preferred the same amino acid residues at P1 (Arg) and P1′ (Ala) positions, respectively. In terms of binding energy contribution usually these are the two most important positions for binding to trypsin-like enzymes. This similarity of the logos showed that the centre of the interaction surface acts similarly in the case of this pair of enzymes. At position P2′ the enzymes selected similar sets of amino acids with some differences in the preference order of the various residues. Both enzymes selected hydrophobic amino acids but the human enzyme preferred the aliphatic side chains while the rat enzyme accepts aromatic side chains (Tyr and Trp) as well. The P3′ and especially the P4′ positions do not seem to contribute to binding energy. In the P3′ and P4′ positions the human enzyme showed a weak preference for positively charged amino acids while the rat enzyme did not show clear amino acid preferences at all.

In the P3 position there is a clear difference between the two logos, although there is an overlapping set of residues at this position too. While the human MASP-2 enzyme preferred the large aromatic Phe and Tyr residues, it also selected smaller residues on a lower scale. On the other hand, rat MASP-2 specifically selected for small hydrophobic amino acids Val, Pro, Ile and Gly at this position.

Based on the logos out of the 6 randomized positions only 3 positions, P3, P1 and P1′ show considerable level of conservation accepting less than 5 residue types in at least one of the species. At P3 we decided to focus on 3 residue types. V and P were chosen as only these two are expected to work well for both species. As we also aimed to test a variant optimal for inhibiting human MASP-2, we chose to test F as well. At P1 R is the single residue to establish high affinity. At P1′ A, G and S are promising to provide strong binding. At P2′ V appears to be best for human, but L seems to be the best common solution. Altogether the small set of 3×1×3×2=18 variants defines the sequence pattern characteristic to inhibit mammalian (i.e. human and/or rat) MASP-2 with high affinity.

The overlapping amino acid preferences of human and rat MASP-2 suggested the existence of a set of inhibitor variants that are effective generally on mammalian MASP-2 enzymes, as these are inhibitors of both the human and the rat enzymes, and MASP-2 enzyme is a highly conserved one among mammals.

In phage display studies, normalized amino acid frequencies usually correlate with binding energy contributions of individual amino acid residues (Pál 2003, 2005a, 2005b, 2006; Rapali 2011; Weiss 2000). On the basis of this notion we designed the presumably tightest human MASP-2 binding TFPI-D2 variant carrying the selected consensus sequence FCRAVKR (SEQ ID NO: 1) between the P3-P4′ positions.

Based on the human and the rat MASP-2 selected sequence logos the apparent human optimum Phe at P3 is likely prohibitive for rat MASP-2 binding. On the other hand, Pro and Val, which are the most preferred residues by rat MASP-2, should be also compatible with human MASP-2 binding, although replacing P3 Phe with any of these residues is expected to decrease binding affinity to the human enzyme. Based on this, two additional variants were designed, namely PCRAVKR (SEQ ID NO: 4) and VCRAVKR (SEQ ID NO: 7) both being P3 variants of SEQ ID NO: 1 having proline and valine at position P3 respectively. In all, the similarity of the sequence logos at the remaining five positions suggested that proteins according to SEQ ID NO: 14 and SEQ ID NO: 17 could be potent inhibitors of both rat and human MASP-2.

We produced these three inhibitors and the parental TFPI-D2 having the inhibitor loop sequence ICRGYIT as recombinant proteins and isolated a greater amount for further tests. The sequence of the inhibitor loops of the three selected clones are shown in Table 3.

TABLE 3
Three most preferred inhibitor loop
sequences according to the invention

| SEQ ID NO: | Sequence |
|---|---|
| 1 | FCRAVKR |
| 4 | PCRAVKR |
| 7 | VCRAVKR |

For production, we used an expression system created by the inventors of the present invention. For more information on it see Example 4.

### Example 4: Heterologous Expression of Inhibitors for Quality Control

All enzymes and reagents were obtained from Fermentas/Thermo Scientific. The reactions were performed according to the company's instructions. During the PCR reactions annealing took place at 50° C. for 30 seconds, 30 cycles were performed with an Esco Swift Mini device. Fermentas/Thermo Scientific GeneJet PCR purification kit (# K0701), Gel extraction kit (# K0691) and Plasmid miniprep kit (# K0502) were used for DNA isolation according to the manufacturer's instructions. All DNA constructs were checked by Sanger sequencing using ABI PRISM BigDye Terminator v3.1 Ready Reaction Cycle Sequencing Kit according to the manufacturer's instructions. The products of the sequencing reactions were analyzed by BIOMI Kft. (Gödöllő). Sequences of the oligonucleotides used in section 4.1. are shown in Table 4 (see later).

### 4.1. Creating the Expression Vectors for the Production of TFPI-D2 Variants

4.1.1. Producing the pS100A4 Expression Vector

The expression vector for the production of the novel TFPI-D2 based MASP-2 inhibitors was made based on the pBH4 plasmid (Kiss 2012). A synthetic gene (purchased from IDT) encoding the N-terminally His6-tagged, C3S, C81S, C86S variant of the human S100A4 protein followed by a TEV protease cleavage site and a multi cloning site was cloned into the vector pBH4 using NcoI and XhoI. The base sequence of the synthetic gene is shown in SEQ ID NO: 28.

This vector was named pS100A4. TFPI-D2 variant genes were cloned into this vector using BamHI and XhoI sites as described in point 4.1.3. and 4.1.4. to obtain a fusion gene construction coding for a fusion protein with the following arrangement:

His6-tag-S100A4-linker peptide-TEV cleavage site-TFPI-D2 variant

The construct enables high-level expression of the fusion proteins in E. coli, purification through immobilized metal ion affinity chromatography through the His6-tag and liberation of TFPI-D2 variants by TEV protease (Tobacco Etch Virus protease) processing.

4.1.2. Cloning the Gene of TFPI-D2 into a Modified pMal Phagemid Vector.

The gene of TFPI-D2 was cloned into a modified pMal p2G phagemid vector to serve as the template in subsequent PCR and mutagenesis reactions. The gene of TFPI-D2 was amplified with PCR using TFPI BamHI forward and TFPI HindIII reverse primers.

In the reaction, the

pTFPI-D2-pro-lib vector served as the template. The PCR product was isolated and eluted with 30 μl 0.1×EB.

The PCR product and the vector were digested with BamHI (10 U) and HindIII (20 U) enzymes in 1× BamHI buffer at 37° C. for 3 hours. The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The TFPI-D2 gene was ligated into the vector using T4 DNA ligase. There was 5-fold molar excess of the PCR product in the ligase reaction.

XL1 Blue cells were transformed with the product of the ligase reaction as described in point 1.3.1.1 and spread on an LB/agar+ampicillin (100 μg/ml) plate. The plate was incubated at 37° C. for 16 hours.

Individual colonies of the transformed XL1 Blue cells were picked into LB+ampicillin (100 μg/ml) and incubated at 37° C. for 16 hours while shaking at 220 rpm. The plasmid DNA was isolated from the cultures. The DNA was eluted from the columns with 50 μl 0.1×EB.

4.1.3. Cloning the Gene of TFPI-D2 into the pS100A4 Vector

The TFPI-D2 gene was amplified with PCR using the pMal 5′ primer and the S100A4 3′ primer pair. The pMal 5′ primer anneals upstream to the coding region of TFPI-D2, while the S100A4 3′ primer anneals downstream from that and introduces an XhoI cleavage site. The PCR product was isolated and eluted with 30 μl 0.1×EB. The PCR product and the pS100A4 vector were digested with BamHI (10 u) and XhoI (20 u) restriction endonucleases at 37° C. for 3 hours to produce the appropriate sticky ends of the PCR product and the vector.

The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The TFPI-D2 gene was ligated into the vector using T4 DNA ligase. There was 5-fold molar excess of the PCR product in the ligase reaction.

XL1 Blue cells were transformed with the product of the ligase reaction as described in point 1.3.1.1. and the cells were spread on an LB/agar+ampicillin (100 μg/ml) plate. The plate was incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

### 4.1.4. Creating the Expression Vectors for the Production of the Variants of the Present Invention

### 4.1.4.1. Creating the Expression Vector for the Production of the Protein of SEQ ID NO: 11

Variant according to SEQ ID NO: 11 was produced by Kunkel mutagenesis as described in point 1.3.1.

CJ236 cells were transformed with the modified pMal p2G phagemid vector containing the gene of TFPI-D2 as described in point 1.3.1.1. The Kunkel mutagenesis was carried out as described in section 1.3.1. using the mutagenesis primer SEQ ID NO: 31 for generating the amino acid sequence of SEQ ID NO: 11 as shown in Table 4.

The verified gene of the amino acid sequence of SEQ ID NO: 11 was amplified with PCR using the pMal 5′ primer (SEQ ID NO: 35) and the S100A4 3′ primer (SEQ ID NO: 34) pair shown in Table 4. The PCR product was isolated and eluted with 30 μl 0.1×EB. The PCR product and the pS100A4 vector were digested with BamHI (10 u) and XhoI (20 u) restriction endonucleases at 37° C. for 3 hours to produce the appropriate sticky ends.

The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The gene of the amino acid sequence of SEQ ID NO: 11 was ligated into the vector using T4 DNA ligase. There was 5-fold molar excess of the PCR product in the ligase reaction.

XL1 Blue cells were transformed with the product of the ligase reaction as described in point 1.3.1.1. and spread on an LB/agar+ampicillin (100 μg/ml) plate. The plate was incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

### 4.1.4.2. Creating the Expression Vector for the Production of Proteins According to SEQ ID NO: 14 and SEQ ID NO: 17

The variants according to SEQ ID NO: 14 and SEQ ID NO: 17 were produced by the two-step megaprimer mutagenesis method using the modified pMal p2G phagemid vector containing the gene of the amino acid sequence according to SEQ ID NO: 11 as template. In the first polymerase chain reaction (PCR) step, an appropriate mutagenesis primer (SEQ ID NO: 32) for the amino acid sequence according to SEQ ID NO: 14, and in a separate PCR an appropriate mutagenesis primer (SEQ ID NO: 33) for the amino acid sequence according to SEQ ID NO: 17 were used in pair with the S100A4 3′ primer (SEQ ID NO: 34), sequences of the primers being listed in Table 4. The products from the two separate PCRs were treated with alkaline phosphatase (FastAP) and exonuclease I (ExoI) enzymes in order to remove residual dNTP's and S100A4 3′ primer. Both reactions were then supplemented with dNTP's, Taq DNA polymerase and pMal 5′ primer and the second PCR step of the megaprimer mutagenesis was performed to generate the full length, mutant PCR products that carry the appropriate flanking restriction endonuclease sites. The PCR products were isolated and eluted with 30 μl 0.1×EB.

The mutant genes were cloned into the pS100A4 fusion expression vector using BamHI and XhoI enzymes. The mutant PCR products and the pS100A4 vector were digested with BamHI (10 U) and XhoI (20 U) in 1× BamHI buffer at 37° C. for 3 hours. The digested DNA products were run on an agarose gel and the fragments of appropriate size were excised and isolated. DNA was eluted from the columns with 30 μl 0.1×EB. The concentrations of the isolated DNA molecules were determined using a BioTek Epoch reader, a Take3 Trio microvolume plate and the Gene5 software. The genes of amino acid sequences according to SEQ ID NO: 14 and SEQ ID NO: 17 were ligated into the vector using T4 DNA ligase. There was 5-fold molar excess of the PCR product in the ligase reaction.

XL1 Blue cells were transformed with the product of the ligase reactions as described in point 1.3.1.1. and spread on an LB/agar+ampicillin (100 μg/ml) plates. The plates were incubated at 37° C. for 16 hours.

Individual colonies of the transformed cells were picked into LB+ampicillin (100 μg/ml) and incubated at 37° C. for 16 hours while shaking at 220 rpm. The plasmid DNA was isolated from the cultures. DNA was eluted from the columns with 50 μl 0.1×EB.

TABLE 4
Sequences of the oligonucleotides used to construct the
variants according to the present invention.
Name and sequence of oligonucleotides used in mutagenesis
and PCR reactions

| | |
|---|---|
| TFPI BamHI forward | AGTAGGATCCAAACCGGACTTCTGC |
| | SEQ ID NO: 29 |
| TFPI HindIII reverse | GTATAAGCTTATTAACCGTCTTCGCAGAT |
| | G |
| | SEQ ID NO: 30 |
| mutagenesis primer for the | CCTGGAAGAAGACCCGGGTTTCTGCCGTG |
| production of the protein | CGGTGAAACGTCGTTACTTCTACAACAAC |
| according to SEQ ID NO: 11 | CAGACC |
| | SEQ ID NO: 31 |
| mutagenesis primer for the | CCTGGAAGAAGACCCGGGTCCGTGCCGTG |
| production of the protein | CGGTGAAACGTCGTTACTTCTACAACAAC |
| according to SEQ ID NO: 14 | CAGACC |
| | SEQ ID NO: 32 |
| mutagenesis primer for the | CGGGTGTGTGCCGTGCGGTGAAACG |
| production of the protein | SEQ ID NO: 33 |
| according to SEQ ID NO: 17 | |
| S100A4 3′ | CTAGTCTCGAGTGCCAAGC |
| | SEQ ID NO: 34 |
| pMal 5′ | GATGTCCGCTTTCTGGTATGC |
| | SEQ ID NO: 35 |

The restriction endonuclease sites are the following:

in SEQ ID NO: 29: from position 5 to 10;
in SEQ ID NO: 30: from position 5 to 10;
in SEQ ID NO: 34: from position 6 to 11.

Bases introducing mutations in mutagenesis reactions are the following:

in SEQ ID NO: 31: from position 20 to 22 and from 29 to 40;
in SEQ ID NO: 32: from position 20 to 22;
in SEQ ID NO: 33: from position 6 to 8.

### 4.1.5. Protein Production

We used E. coli Shuffle T7 (NEB, C3026H) for protein expression. This strain was engineered to allow the formation of disulfide bridges in the cytoplasm. It also expresses the disulfide bond isomerase and chaperone protein DsbC in the cytoplasm to help protein folding by assisting in the formation of the most stable native disulfide bridge pattern (Lobstein 2012).

### Transformation

1 μl expression vector
100 μl Shuffle T7 competent cell
The cells were incubated on ice for 30 minutes, and then for 1 minute they were exposed to a heat shock at 42° C.
200 μl LB medium was added to the cells, it was shaken for 30 minutes at 37° C., and then it was spread on an LB/agar+ampicillin (100 μg/ml) plate. The plate was incubated overnight at 30° C.

### Production

Cells on the plate were washed into 30 ml LB+ampicillin (100 μg/ml) and shaken at 30° C. to serve as the initial culture. Two to three litres of LB was poured into 2.8 l Fernbach flasks (1 l LB in each flask) and supplemented with ampicillin to 100 μg/ml final concentration. The initial culture was dispensed amongst the Fernbach flasks equally and the flasks were incubated at 30° C. while shaking at 180 rpm until the cultures reached the value OD_{600 nm}=0.8. It took about 4-6 hours depending on the density of the initial culture and the total volume of expression. At this point we induced the cells with an IPTG solution of a final concentration of 0.4 mM, and shook it for 14-16 more hours at 30° C. Then the cells were centrifuged (5 minutes, 7,500 g, 4° C.), the supernatant was poured off and the cells were suspended in 1/10 culture volume of 50 mM Tris-HCl, 300 mM NaCl, 10 mM imidazole

pH 8.0 buffer.

### Purification

The cells were disrupted by sonication and the samples were centrifuged to remove the cell debris (20 minutes, 48,000 g). The supernatant containing the fusion protein and other soluble components of the cytoplasm was loaded onto an IMAC column (15 ml BioRad Profinity IMAC resin) containing immobilized nickel ions. The column was equilibrated with 50 mM Tris-HCl, 300 mM NaCl, 10 mM imidazole pH 8.0 buffer (chromatography buffer). The column was washed with 20 column volume of chromatography buffer after loading the sample. The His-tagged S100A4-fused inhibitors were eluted with chromatography buffer supplemented with 250 mM imidazole.

The eluted fusion protein was dialyzed against chromatography buffer in order to reduce the concentration of imidazole in the sample using dialysis tubing cellulose membrane with a cut-off value of 12-14 kDa (Sigma—D9527).

### Processing

TEV protease cleavage took place under the following conditions:

### 50 mM TrisHCl pH 8.0

### 300 mM NaCl

250 μM beta-mercaptoethanol
˜50 μg/mL TEV protease

The reaction was incubated at 30° C. for 16 hours. We produced the TEV protease ourselves on the basis of the publication of van den Berg, 2006, with modifications. For cleavage, we did not add a reducing agent to the solution to protect the disulphide bridges of the TFPI-D2 variants. The beta-mercaptoethanol present in the solution derives from the storage buffer of the TEV protease. The cleavage was tested with 15% SDS PAGE method.

### Isolation of TFPI-D2 Variants

In this phase, in the solution there are the inhibitor (TFPI-D2 variant), His6-tagged S100A4, His6-tagged TEV protease and possibly some unprocessed fusion protein. The sample was centrifuged to remove any precipitations and reloaded onto the IMAC column equilibrated with chromatography buffer. The His6-tagged proteins remained attached to the immobilized nickel ions on the resin while the processed inhibitor was in the flow through. The inhibitor was isolated using reversed-phase HPLC procedure, on a Phenomenex Jupiter C4 300A type, 250×10 mm semi-preparative column. The sample was filtered through a 0.22 um sterile filter, and then it was taken to a column equilibrated with 0.1% trifluoroacetic acid (TFA)/distilled water solution (solution A). For separation, we used acetonitrile (HPLC grade)/0.1% TFA solution (solution B). The gradient was 1%/minute between 20% and 40%. The eluent flow rate was 2 ml/minute. The inhibitor variants eluted between 27-32% solution B, depending the amino acid sequence. Besides 220 nm the process could also be detected with 280 nm UV light absorption, as all clones produced by us contained Tyr side chains. Separation was realised with HP1100 type HPLC system. Agilent ChemStation software was used for system control, data collection and evaluation.

### Quality Control—HPLC/MS

In the case of all isolated inhibitors mass spectrometry was used for quality control. Mass spectrometry analysis was realised with HP1100 type HPLC-ESI-MS system, with flow-injection method, using 10 mM ammonium formate, pH 3.5 solution. The settings of the device were the following. Both the drying and the pulverising gas was nitrogen, the flow rate of the drying gas was 10 l/minute, its temperature was 300 Celsius degrees. The pressure of the pulverising gas was 210 kPa, the capillary voltage was 3500 V. The total ion current (TIC) chromatogram was recorded in positive ion setting within the range of 100-1500 mass/charge. The mass data were evaluated with Agilent ChemStation software.

The sequences of the individual inhibitors produced and the sequence of the inhibitor loop and mass data are included in Table 5.

TABLE 5
The theoretical and measured molecular weights of
a few inhibitors produced, according to the
invention

| SEQ ID | Sequence of the | Theoretical | Measured |
|---|---|---|---|
| NO: | inhibitor loop | weight (Da) | weight (Da) |
| 11 | FCRAVKR | 7038.8 | 7038.8 |
| 14 | PCRAVKR | 6988.8 | 6988.7 |
| 17 | VCRAVKR | 6990.8 | 6990.8 |
| 23 | ICRGYIT | 6984.7 | 6984.9 |

### 4.2. Determining K_{I} Constants on MASP Enzymes and Bovine Trypsin

The inhibition equilibrium constant (K_{I}) of all four inhibitor variants produced was measured on human MASP-2 and rat MASP-2. Additionally, K_{I} values were measured also on human MASP-1 and human MASP-3 to assess the specificity of the inhibitors amongst the MASP enzymes. K_{I} values of the inhibitors on bovine trypsin, a pancreatic model enzyme was also measured.

For determining the K_{I} of the inhibitors on MASP enzymes we used catalytic enzyme fragments containing the three C-terminal domains: CCP1-CCP2-SP. The synthetic substrate used in the measurements was Z-L-Lys-SBzl hydrochloride (Sigma, C3647), from which a 10 mM stock solution was prepared. The reactions were performed in a volume of 0.2 ml at room temperature in a buffer consisting of 20 mM HEPES; 145 mM NaCl; 5 mM CaCl₂; 0.05% Triton-X100. The substrate cleaved by the enzyme entered into a reaction with the 5,5′-Dithiobis(2-nitrobenzoic acid) (DTNB, Ellman's reagent, Sigma—D8130) auxiliary substrate present in the solution in 2-fold excess to Z-L-Lys-SBzl. The release of the chromophore group created in this way was monitored via the change of absorbance at 410 nm using a BioTek Synergy H4 multimode microplate reader.

A dilution sequence was prepared from the individual inhibitors, the enzyme was added to it, and it was incubated for 2 hours at room temperature. The samples were transferred on a 96-well microtiter plate (Nunc 269620). The reactions were started by adding the mixture of the substrate and the auxiliary substrate to the samples. The concentration of the substrate and the length of the measuring period were chosen so that under the given conditions the enzyme should consume less than 10% of the substrate. In this case we used 250 μM Z-L-Lys-SBzl and 500 μM DTNB in the reactions. In the course of measuring, a method developed for the characterisation of tight-binding inhibitors was used (Empie, 1982). The concentration of the product was measured as a function of reaction time. The slope of the straight line determined for the initial phase of the reaction was normalised with the slope obtained for the uninhibited enzyme reaction, and this value was multiplied with the total enzyme concentration. As a result of this we obtained the free enzyme concentration, which was plotted as a function of the total inhibitor concentration and the Ki value was determined according to the following equation:

E=y=E₀−(E₀+x+Ki−(((E₀+x+Ki)\hat{ }2)−4*E₀*x)\hat{ }(½))/2,

where E is the free (uninhibited) enzyme concentration, and E₀ is the total enzyme concentration. The stock concentration of the inhibitors was determined by titration with bovine trypsin of known concentration. The results were calculated as the average of parallel measurements. The results are summarised in Table 6 below.

The K_{I} values of the inhibitors on bovine trypsin were also determined. In these experiments, we used bovine trypsin (Sigma—T1426). The concentration of trypsin was determined by fluorescent active-site titration based on Jameson 1973. The measurements were done in 200 μL final assay volume of 50 mM Tris, 10 mM CaCl2, 0.005% Triton X-100 pH 8.0 buffer on 96-well black microtiter plates (Thermo Scientific Sterilin—611F96BK) using a BioTek Synergy H4 multimode microplate reader.

Constant predefined concentrations of the enzymes were mixed with serial dilutions of the inhibitors and co-incubated at room temperature until reaching equilibrium. The incubation times were 5 h in the case of the protein according to SEQ ID NO: 11, 8 h in the case of TFPI-D2 and the protein according to SEQ ID NO: 17 and 24 h in the case of the protein according to SEQ ID NO: 14. Then, 10 μM (final concentration) of Z-Gly-Pro-Arg-AMC substrate was added to the samples. The concentration of the free enzyme was determined through measuring the residual enzyme activity in a fluorometric kinetic assay at 380 nm excitation wavelength and 460 nm emission wavelength in two parallel experiments. Analyses of the data were done as described above. The K_{I} values are shown in Table 6.

TABLE 6
Summarising the enzyme inhibition potencies of the
individual inhibitors in terms of equilibrium inhibition
constants (K_{I}).

| | Sequence | K_{I} values (nM) of various enzyme/inhibitor | | | | |
|---|---|---|---|---|---|---|
| SEQ | of the | combinations | | | | |
| ID | inhibitor | human | | human | human | |
| NO: | loop | MASP-2 | rat MASP-2 | MASP-1 | MASP-3 | bovine trypsin |
| 11 | FCRAVKR | 2.0 ± 0.1^{b} | 640 ± 20^{b} | N.E. | N.E. | 0.094 ± 0.001^{b} |
| 14 | PCRAVKR | 7.9 ± 0.3^{b} | 7.2 ± 0.2^{a} | N.E. | 30 000^{c} | 0.004 ± 0.0001^{b} |
| 17 | VCRAVKR | 36.7 ± 0.7^{b} | 7.5 ± 0.3^{b} | N.E. | N.E. | 0.017 ± 0.0002^{b} |
| 23 | (TFPI-D2) | 1883 ± 48^{b} | 185 ± 4^{b} | N.E. | N.E. | 0.015 ± 0.0005^{b} |
| | ICRGYIT | | | | | |
| 36 | SGMI-2 | 6^{d} | 22.7 ± 1.6^{a} | N.E. | ~5000^{e} | 100 ± 7^{a} |
| | VCTKLWCN | 8.1 ± 0.3^{b} | | | | |

^{a}Average ± SD (n = 3) is indicated
^{b}Average ± range (n = 2) is indicated
^{c}Approximation based on a single measurement
^{d}Data from Héja et al. 2012b
^{e}Data from Héja et al. 2012a
N.E.: not effective

The results show that the variants according to the present invention are potent inhibitors of human MASP-2. The K_{I} values are between 2-37 nM. The protein according to SEQ ID NO: 11 carrying the human MASP-2 selected consensus sequence on its inhibitory loop is the tightest human MASP-2 binder of the set with a K_{I} value of 2.0 nM. The proteins according SEQ ID NO: 14 and SEQ ID NO: 17 have K_{I} values of 7.9 nM and 36.7 nM, respectively. This means that the substitution of the optimal P3 phenylalanine with proline or valine results in 4-fold or 18-fold reduction, respectively in terms of binding affinity to human MASP-2. This is in good agreement with the relative frequencies of these amino acid residues in the selected pool of clones as illustrated by the corresponding sequence logo.

The protein according SEQ ID NO: 11 with its 2 nM K_{I} value is 3-fold more potent than SGMI-2 (SEQ ID NO: 36, described in WO 2012/007777), the tightest binder human MASP-2 inhibitor developed previously based on the SGCI inhibitor scaffold (Héja 2012b).

It was shown previously in a solid phase complement ELISA assay using diluted serum (Wouters 2011; Keizer 2015) that TFPI-D2 inhibits MASP-2. The reported inhibitory effect is extremely weak (IC50=10 μM) and the physiological relevance of this interaction should be negligible. Nevertheless, we determined the K_{I} values of the TFPI-D2-MASP-2 interactions in part to see the extent of affinity improvement achieved by the protein variants of the present invention. We found that human TFPI-D2 is a weak inhibitor of human MASP-2

(K_{I}=1883 nM) and a moderate inhibitor of rat MASP-2

### (K_{I}=185 nM).

The protein variants of the present invention therefore represent 50-940-fold improvements compared to TFPI-D2 in terms of binding affinity for human MASP-2.

Between the P1-P4′ positions of the inhibitory loop, the protein variants of the present invention carry the human MASP-2 selected consensus sequence. Based on the sequence logos (see FIGS. 3.a and 3.b) these side chains should be preferred or at least acceptable for binding to rat MASP-2 as well. On the other hand, at position P3 there is an obvious difference between the set of residues preferred by the human and rat enzymes. Human MASP-2 prefers the large aromatic Phe and Tyr residues, while rat MASP-2 prefers small aliphatic residues such as Val, Pro or Ile. This apparent difference in preferences was fully verified by the inhibitory experiments. While the protein according to SEQ ID NO: 14 having a P3 Pro and the protein according to SEQ ID NO: 17 having a P3 Val are potent inhibitors of rat MASP-2 with K_{I} values of 7.2 nM and 7.5 nM, respectively, the protein according SEQ ID NO: 11 having a P3 Phe is only a very weak inhibitor of rat MASP-2 with a K_{I} value of 640 nM. On rat MASP-2, binding affinities of the protein variants according to SEQ ID NO: 14 and SEQ ID NO: 17 are 25-fold higher than that of human TFPI-D2. The K_{I} value of SGMI-2 determined on rat MASP-2 is 22.7 nM. Based on these results the proteins according to SEQ ID NO: 14 and SEQ ID NO: 17 are about 3-fold more potent inhibitors of rat MASP-2 than SGMI-2.

No considerable inhibition of MASP-1 or MASP-3 was detected with TFPI-D2 and the protein variants mentioned above.

K_{I} values of TFPI-D2 and the protein variants of the present invention on bovine trypsin were also measured. The K_{I} values are shown in Table 5 above. The parental molecule, TFPI-D2 inhibits bovine trypsin with a K_{I} value of 15 pM. The high affinity of TFPI-D2 towards bovine trypsin is in good agreement with previous data in the literature: it was reported that TFPI-D2 has a K_{I} value of 100 pM towards porcine trypsin, an enzyme highly similar to bovine trypsin (Petersen 1996). The protein variants of the present invention have K_{I} values in the 4-94 pM range towards bovine trypsin. The protein according to SEQ ID NO: 14 is the strongest trypsin-binder; the protein according SEQ ID NO: 11 is the weakest one, while the affinity of the protein according SEQ ID NO: 17 is similar to that of TFPI-D2. These data show that the inhibitor loops of the protein variants of the present invention evolved to be optimal for inhibiting human and/or rat MASP-2 also enable high-affinity bovine trypsin inhibition. This high affinity towards bovine trypsin allows for accurate determination of the inhibitor concentration by titration against bovine trypsin of known concentration.

### 4.3. The Effects of the Protein Variants of the Present Invention on the Three Complement-Activation Pathways in Diluted Human Serum

As outlined above, the complement system can be activated through three pathways, which lead to the same single end-point. The three activation pathways are the classical, the lectin and the alternative pathway. The MASP enzymes belong strictly to the lectin pathway and MASP-2 is a key enzyme of the lectin pathway activation. The protein inhibitors of the present invention were therefore expected to block lectin pathway activation while not affecting the other two pathways or the convertase enzymes of the common complement route.

The so-called WIELISA kit (Euro-Diagnostica AB, COMPL300) was developed for selective measurement of the activation of the three complement pathways. By following the instructions of the kit this assay was used for assessing the inhibitory potency of the protein inhibitors according to SEQ ID NO: 11, SEQ ID NO: 14 and SEQ ID NO: 17 on each pathway. The kit applies three different conditions, each ensuring that only one of the three pathways can be activated, while the other two remain inactive. The kit detects the latest emerging component of complement activation on the route where the three pathways already merged: a neo-epitope of C9 in the C5-9 complex.

The assay was performed using normal human serum (NHS) pooled from at least 10 healthy individuals. The blood samples were incubated for 1 hour at room temperature, then centrifuged, the serum fraction was mixed and stored in small aliquots at −80° C. Normal human serum was thawed on ice and diluted with the provided buffers of the kit according to the instructions of the manual. The dilution of the serum was 50-fold in the case of the classical and lectin pathway measurements and 9-fold in the case of the alternative pathway measurement. The diluted samples were incubated at room temperature for 20 minutes. Serial dilutions of the inhibitors were made in the provided buffers and were added to the diluted serum samples to reach final serum dilutions 100-fold in the case of the classical and lectin pathway measurements and

18-fold in the case of the alternative pathway measurement. The samples were incubated for another 20 minutes at room temperature and then transferred to the WiELISA plate. The plate was incubated at 37° C. for 60 minutes, washed with the provided washing buffer and 100-100 μl of the provided conjugate was pipetted into the wells. The plate was incubated at room temperature for 20 minutes. The plate was washed again and 100-100 μl of the provided substrate solution was pipetted into the wells. After signal development 100 μl/well 5 mM EDTA solution was used to stop the reaction and the plate was read at 405 nm using a PerkinElmer EnSpire multimode plate reader. Two parallels were measured for each data point.

100% activity was represented by the serum without any inhibitor added. The measurements were performed at the same time and on the same plate, from one single thawed serum sample.

In this experiment, we measured the complement inhibitory effects of the protein variants according to the present invention and SGMI-2. It was because we aimed to directly compare the efficacy of the protein variants of the present invention to our previous, second generation MASP-2 inhibitors disclosed in WO 2012/007777, and also because IC₅₀ values should depend on the actual serum sample used.

The measurements demonstrated that the protein variants of the present invention are efficient and specific inhibitors of the lectin pathway of the complement system. The IC₅₀ values are in the 10⁻⁷-10⁻⁸ M range and show that the protein according to SEQ ID NO: 11 and SEQ ID NO: 14 are more efficient inhibitors of the lectin pathway than SGMI-2. The IC₅₀ value of the protein according to SEQ ID NO: 17 is slightly (2-fold) higher than that of SGMI-2 (WO 2012/007777). This result is in compliance with the result demonstrated earlier, according to which these inhibitors inhibit the MASP-2 enzyme very efficiently, which enzyme, according to our present knowledge, is essential for the initiation of the lectin pathway.

The inhibitor concentrations (IC₅₀) needed for reducing the uninhibited lectin pathway activity by half are included in Table 7.

TABLE 7
The inhibitor concentrations (IC₅₀) needed for
reducing the uninhibited lectin pathway activity
by half in diluted human serum and comparison to
the K_{I} values measured on purified human MASP2cf.

| SEQ | | | | |
|---|---|---|---|---|
| ID | Sequence of the | IC₅₀ | K_{I} on human | |
| NO: | inhibitor loop | (nM) | MASP-2 (nM) | IC₅₀/K_{I} |
| 11 | FCRAVKR | 35 | 2.0 | 17.5 |
| 14 | PCRAVKR | 90 | 7.9 | 11.4 |
| 17 | VCRAVKR | 380 | 36.7 | 10.4 |
| 36 | SGMI-2 | 174 | 6^{a} | 29 |
| | VCTKLWCN | | | |

^{a}Data based on Héja et al., 2012b

Numerous serine proteases operate in the complement system, and some of them are very similar to the MASP enzymes. Despite this the MASP-2 inhibitors according to the present invention did not inhibit at all either the classical or the alternative pathway up to the final concentration of 10 μM in the diluted human serum.

As in the course of measuring the classical and the alternative pathway the presence of the inhibitors did not inhibit the creation of the terminal C5-9 complex, it is for certain that the inhibitors do not inhibit the proteases of the joint section of the complement system, so the inhibition of the lectin pathway really took place at the beginning of the lectin pathway, at the level of the MASP enzymes. It is worth pointing out that the IC₅₀ data obtained in the course of the WIELISA measurement is about 10-30 times higher than the K_{I} values obtained in the course of MASP-2 inhibition measurements based on synthetic substrates. A possible explanation for this is the following. The inhibitors bind to the MASP-2 enzyme directly at the substrate binding site, and this binding successfully competes with the relatively weak interaction of small synthetic substrates with the same enzyme surface. However, besides the substrate binding site situated on the protease domain, physiological substrates can create bonds via other surfaces too (exosites), and they bind to the enzyme with a higher affinity than small synthetic substrates. It is because of this higher affinity that inhibitor molecules must be used in a higher concentration for the balance to be shifted from the enzyme-substrate complex towards the enzyme-inhibitor complex.

It is a fact of outstanding importance that, as compared to the SGCI-based SGMI-2 inhibitor, significantly lower inhibitor concentrations were sufficient to reduce the pathway activity by half in the case of inhibitors according to SEQ ID NO: 11 and SEQ ID NO: 14. This may have a great practical significance in the course of experiments performed later on living systems, where a typical task may involve setting the inhibitor concentration inside the entire vascular system of a living organism so that the given MASP enzyme is completely inhibited.

### 4.4. The Effect of the Protein of SEQ ID NO: 14 on the Lectin Pathway in Rat Serum

ELISA-based complement lectin pathway activation tests were performed on mannan coated plates using diluted individual (not pooled) rat serum. In these tests, inhibitory efficiencies of the protein of SEQ ID NO: 14 and SGMI-2 were compared. Three different assays were conducted that detect the deposition of C3, C4 or the terminal complement antigen C5b-9, respectively.

4.4.1. C3 Deposition ELISA with Diluted Rat Serum

96-well Greiner high binding ELISA plates (cat. no. 655061) were coated with 100 μl/well 10 μg/ml mannan dissolved in coating buffer (50 mM sodium-carbonate pH 9.6) overnight at 4° C. Control wells contained coating buffer alone. Wells were blocked for at least 1 h at 37° C. with 200 μl/well

10 mg/ml bovine serum albumin (BSA) dissolved in 50 mM Tris pH 7.4, 150 mM NaCl, 0.1% Tween-20 buffer. Serial dilutions of the inhibitors were made in 10 mM HEPES pH 7.4, 150 mM NaCl, 5 mM CaCl₂, 5 mM MgCl₂, 0.1% Tween-20 buffer (serum dilution buffer). Rat serum was thawed on ice and diluted 35-fold in serum dilution buffer. The diluted inhibitor samples were mixed with the 35-fold diluted rat serum in 1:1 volume ratio resulting in 70-fold diluted rat serum-inhibitor samples. The samples were incubated at room temperature for 30 minutes. The plate was rinsed with

50 mM Tris pH 7.4, 150 mM NaCl, 5 mM CaCl₂, 0.1% Tween-20 buffer (washing buffer) and 100 μl of the serum-inhibitor samples were transferred onto the plate. 70-fold diluted rat serum (containing no inhibitor) was transferred onto mannan coated surfaces as positive control to assess maximal complement activity. Two negative controls were made. In one, diluted rat serum was transferred on surfaces treated only with BSA, while in the other one diluted rat serum supplemented with 50 μM FUT-175 (a broad specificity serine protease inhibitor) (Sigma—N0289) was transferred onto mannan coated wells. The plate was incubated at 37° C. for 30 minutes and then rinsed with washing buffer. 100 μl/well α-human C3c polyclonal antibody (rabbit) (DakoCytomation—A0062) diluted 2000-fold in 50 mM Tris pH 7.4, 150 mM NaCl, 5 mM CaCl₂, 1% BSA, 0.1% Tween-20 buffer was transferred onto the plate and the plate was incubated at 37° C. for 1 hour. The rabbit α-human-C3c polyclonal antibody recognizes rat C3. After washing, 100 μl/well peroxidase conjugated α-rabbit IgG monoclonal antibody (mouse) (Sigma—A1949) diluted 40 000-fold in 50 mM Tris pH 7.4, 150 mM NaCl, 5 mM CaCl₂, 1% BSA, 0.1% Tween-20 buffer was transferred to the plate and the plate was incubated for 30 minutes at 37° C. The plate was rinsed again with washing buffer. Then, 100 μl/well 1 mg/ml o-phenylenediamine dihydrochloride (OPD, Sigma—P9029) peroxidase substrate dissolved in 50 mM citrate pH 5.0, 0.05% H₂O₂ buffer was transferred to the plate to generate a photometric signal proportionate to the amount of C3 deposited onto the surface. After signal development, the reaction was stopped by adding 50 μl/well 1 M sulfuric acid. The 490 nm absorbance values were recorded using a PerkinElmer EnSpire multimode plate reader. Four parallels were measured for each data point.

4.4.2. C4 Deposition ELISA Assay with Diluted Rat Serum

The assay was performed on mannan coated ELISA plates as in the case of C3 deposition with diluted rat serum. The final dilution of rat serum was 70-fold. Rabbit α-human-C4c polyclonal antibody (DakoCytomation—Q0369) was used as the primary antibody in 2 000-fold dilution. It recognizes rat C4. 40 000-fold diluted peroxidase conjugated α-rabbit IgG monoclonal antibody (mouse) was used as secondary antibody.

4.4.3. C5b-9 Deposition ELISA Assay with Diluted Rat Serum

The assay was performed on mannan coated ELISA plates as in the case of C3 deposition with diluted NHS. The final dilution of rat serum was 50-fold. Monoclonal mouse α-rat C5b-9 antibody (Santa Cruz Biotechnology—sc-66190) was used as the primary antibody in 1000-fold dilution. Peroxidase conjugated anti-mouse antibody (AbCam—ab97265) was used as secondary antibody in 3000-fold dilution.

Both the protein of SEQ ID NO: 14 and SGMI-2 were able to completely inhibit the lectin pathway in rat serum. The IC₅₀ values are shown in Table 8. In all three assays, the protein of SEQ ID NO: 14 is more efficient than SGMI-2 having IC50 values 2.5-4.2-fold lower than SGMI-2. The K_{I} value of the protein of SEQ ID NO: 14 in in vitro assays with rat MASP-2 is 7.2 nM which is 3-fold lower than the 22.7 nanomolar K_{I} value of SGMI-2 against rat MASP-2. The ratio of the IC₅₀ values of the two inhibitors in the complement deposition ELISA assays is in good agreement with the ratio of the K_{I} values of the inhibitors.

Based on these results compared to SGMI-2, the protein of SEQ ID NO: 14 is significantly more effective lectin pathway inhibitor in rat serum as well.

In all, the protein of SEQ ID NO: 14 is a potent inhibitor of both the human as well as the rat lectin pathway.

TABLE 8
The inhibitor concentrations (IC₅₀) needed for
reducing the uninhibited lectin pathway activity
by half in diluted rat serum tests.

| | IC₅₀ (nM) | |
|---|---|---|
| Name | the protein | |
| (Sequence of the | of SEQ ID NO: | SGMI-2 |
| inhibitor loop) | 14(FCRAVKR) | (VCTKLWCN) |
| C3 deposition | 869 | 2239 |
| C4 deposition | 108 | 457 |
| C5b-9 deposition | 940 | 4000 |

### 4.5. The Effect of the Protein of SEQ ID NO: 14 on the Classical and Alternative Pathways in Rat Serum

ELISA-based complement classical and alternative pathway activation tests were performed on IgG or lipopolysaccharide (LPS) coated plates, respectively, using diluted individual (not pooled) rat serum. In these tests, inhibitory efficiencies of the protein of SEQ ID NO: 14 and SGMI-2 were compared and C3 deposition was used for quantification of the complement activation.

4.5.1. C3 Deposition ELISA with Diluted Rat Serum on IgG Coated Plates

96-well Greiner high binding ELISA plates were coated with 100 μl/well 10 μg/ml IgG (Sigma—12511) dissolved in coating buffer (50 mM sodium-carbonate pH 9.6) overnight at 4° C. Control wells contained coating buffer alone. Wells were blocked for 2 h at 37° C. with 200 μl/well 10 mg/ml bovine serum albumin (BSA) dissolved in 50 mM Tris pH 7.4, 150 mM NaCl, 0.1% Tween-20 buffer. Serial dilutions of the inhibitors were made in 10 mM HEPES pH 7.4, 150 mM NaCl, 5 mM CaCl₂, 5 mM MgCl₂, 0.1% Tween-20 buffer (serum dilution buffer). Rat serum was thawed on ice and diluted 25-fold in serum dilution buffer. The diluted inhibitor samples were mixed with the 25-fold diluted rat serum in 1:1 volume ratio resulting in 50-fold diluted rat serum-inhibitor samples. The samples were incubated at room temperature for 30 minutes. The plate was rinsed with 50 mM Tris pH 7.4, 150 mM NaCl, 5 mM CaCl₂, 0.1% Tween-20 buffer (washing buffer) and 100 μl of the serum-inhibitor samples were transferred onto the plate. 50-fold diluted rat serum (containing no inhibitor) was transferred onto IgG coated surfaces as positive control to assess maximal complement activity. Two negative controls were made. In one, diluted rat serum was transferred on surfaces treated only with BSA, while in the other one diluted rat serum supplemented with 50 μM FUT-175 (a broad specificity serine protease inhibitor) (Sigma—N0289) was transferred onto IgG coated wells. The plate was incubated at 37° C. for 30 minutes and then rinsed with washing buffer. 100 μl/well α-human C3c polyclonal antibody (rabbit) (DakoCytomation—A0062) diluted 2000-fold in 50 mM Tris pH 7.4, 150 mM NaCl, 5 mM CaCl₂, 1% BSA, 0.1% Tween-20 buffer was transferred onto the plate and the plate was incubated at 37° C. for 1 hour. The rabbit α-human-C3c polyclonal antibody recognizes rat C3. After washing, 100 μl/well peroxidase conjugated α-rabbit IgG monoclonal antibody (mouse) (Sigma—A1949) diluted 40,000-fold in 50 mM Tris pH 7.4, 150 mM NaCl, 5 mM CaCl₂, 1% BSA, 0.1% Tween-20 buffer was transferred to the plate and the plate was incubated for 30 minutes at 37° C. The plate was rinsed again with washing buffer. Then, 100 μl/well 1 mg/ml o-phenylenediamine dihydrochloride (OPD, Sigma—P9029) peroxidase substrate dissolved in 50 mM citrate pH 5.0, 0.05% H₂O₂ buffer was transferred to the plate to generate a photometric signal proportionate to the amount of C3 deposited onto the surface. After signal development, the reaction was stopped by adding 50 μl/well 1 M sulfuric acid. The 490 nm absorbance values were recorded using a PerkinElmer EnSpire multimode plate reader. Four parallels were measured for each data point.

4.5.2. C3 Deposition ELISA with Rat Serum on LPS Coated Plates

The buffers, incubation times, incubation temperatures and antibodies were the same as described in point 4.5.1.

The final dilution of the rat serum was 6-fold. The dilution of the primary antibody was 3000-fold.

The results show that neither the protein of SEQ ID NO: 14 nor SGMI-2 were able to inhibit the classical or alternative pathway in rat serum at all even when added in final concentrations as high as 50 μM.

Based on these results the protein of SEQ ID NO: 14 is a highly selective inhibitor of the lectin pathway of complement in both human and rat serum.

### 4.6. The Effect of the Protein Variants of the Present Invention on Blood Coagulation

Blood coagulation measurements using blood plasma taken from healthy human individuals were also performed. From the blood obtained through venipuncture and treated with sodium citrate (3.8% wt/vol) the plasma was isolated by centrifugation (2,000 g, 15 minutes, Jouan CR412 centrifuge).

The effect of the protein variants of the present invention on the blood coagulation process was tested in three standard assays, the thrombin time, testing any direct effects on thrombin; prothrombin time, testing any effects on the extrinsic pathway; and the activated partial thromboplastin time, testing any effects on the intrinsic pathway. Blood was collected from a healthy individual by vein puncture after informed consent. The blood was treated with sodium-citrate (3.8% w/v) and centrifuged. All three assays were performed on the automated instrument Sysmex CA-1500 (Sysmex) with Innovin reagent (Dale Behring, Marburg, Germany).

The inhibitors were applied in a fivefold serial dilution with the highest final concentration being 36 μM. This value is 3-4 orders of magnitudes higher than the K_{I} values of the three protein variants of the present invention on human MASP-2. Even at the highest concentration the protein variants of the present invention have only little effect in the APTT test and no effect in the PT and TT tests. The corresponding blood clotting times in seconds are shown in Table 7.

On the basis of the results it can be clearly stated about the protein variants of the present invention that they do not inhibit any of the six blood coagulation proteases: thrombin, fVIIa, fIXa, fXa, fXIa and fXIIa with considerable affinity. It complies with our knowledge according to which MASP-2 and blood coagulation proteases have no common physiological substrate. The results are shown in Tables 9.a, 9.b, and 9.c.

TABLE 9
The effect of the protein inhibitors of the
present invention on blood coagulation times.
a

| | APTT (s) | | |
|---|---|---|---|
| [inhibitor](μM) | SEQ ID NO: 11 | SEQ ID NO: 14 | SEQ ID NO: 17 |
| 0.0 | 36.4 | 36.5 | 36.6 |
| 0.1 | 36.3 | 36.1 | 36.8 |
| 0.3 | 36.5 | 36.8 | 37.2 |
| 1.4 | 37.0 | 37.9 | 38.1 |
| 7.2 | 39.3 | 40.2 | 40.6 |
| 36.0 | 46.0 | 46.4 | 47.4 |

b

| | PT (s) | | |
|---|---|---|---|
| [inhibitor](μM) | SEQ ID NO: 11 | SEQ ID NO: 14 | SEQ ID NO: 17 |
| 0.0 | 10.9 | 10.8 | 10.7 |
| 0.1 | 10.7 | 10.8 | 10.8 |
| 0.3 | 10.8 | 10.7 | 10.8 |
| 1.4 | 11.0 | 10.8 | 10.8 |
| 7.2 | 11.0 | 10.9 | 10.9 |
| 36.0 | 11.4 | 11.3 | 11.4 |

c

| | TT (s) | | |
|---|---|---|---|
| [inhibitor](μM) | SEQ ID NO: 11 | SEQ ID NO: 14 | SEQ ID NO: 17 |
| 0.0 | 16.7 | 16.5 | 16.4 |
| 0.1 | 16.5 | 16.5 | 16.6 |
| 0.3 | 16.6 | 16.6 | 16.3 |
| 1.4 | 16.7 | 16.7 | 16.4 |
| 7.2 | 16.4 | 16.2 | 16.4 |
| 36.0 | 16.0 | 15.5 | 16.0 |

### LITERATURE REFERENCES

Ambrus, G., Gál, P., Kojima, M., Szilágyi, K., Balczer, J., Antal, J., Gráf, L., Laich, A., Moffatt, B., Schwaeble, W., Sim, R. B. and Závodszky, P. (2003) Natural substrates and inhibitors of mannan-binding lectin-associated serine protease-1 and -2: a study on recombinant catalytic fragments. J. Immunol. 170, 1374-1382.
Ammitzboll, C. G., Thiel, S., Ellingsen, T., Deleuran, B., Jorgensen, A., Jensenius, J. C. and Stengaard-Pedersen, K. (2012) Levels of lectin pathway proteins in plasma and synovial fluid of rheumatoid arthritis and osteoarthritis. Rheumatol. Int. 32, 1457-1463.
Arlaud, G. J., Gaboriaud, C., Thielens, N. M., Budayova-Spano, M., Rossi, V. and Fontecilla-Camps, J. C. (2002) Structural biology of the C1 complex of complement unveils the mechanisms of its activation and proteolytic activity. Mol. Immunol. 39, 383-394.
Asgari, E., Farrar, C. A., Lynch, N., Ali, Y. M., Roscher, S., Stover, C., Zhou, W., Schwaeble, W. J. and Sacks, S. H. (2014) Mannan-binding lectin-associated serine protease 2 is critical for the development of renal ischemia reperfusion injury and mediates tissue injury in the absence of complement C4. FASEB J. 28(9):3996-4003.
Beinrohr, L., Dobó, J., Závodszky, P. and Gál, P. (2008) C1, MBL-MASPs and C1-inhibitor: novel approaches for targeting complement-mediated inflammation. Trends in Molecular Medicine doi:10.1016/j.molmed.2008.09.009.
van den Berg, S., Löfdahl, P. A., Hard, T. and Berglund, H. (2006) Improved solubility of TEV protease by directed evolution. Journal of Biotechnology 121, 291-298.
Bora, N. S., Jha, P. and Bora, P. S. (2008) The role of complement in ocular pathology. Semin. Immunopathol. 30, 85-95.
Burk, A. M., Martin, M., Flierl, M. A., Rittirsch, D., Helm, M., Lampl, L., Bruckner, U., Stahl, G. L., Blom, A. M., Perl, M., Gebhard, F. and Huber-Lang, M. (2012) Early complementopathy after multiple injuries in humans. Shock. 37, 348-354.
Castellano, G., Melchiorre, R., Loverre, A., Ditonno, P., Montinaro, V., Rossini, M., Divella, C., Battaglia, M., Lucarelli, G., Annunziata, G., Palazzo, S., Selvaggi, F. P., Staffieri, F., Crovace, A., Daha, M. R., Mannesse, M., van Wetering, S., Paolo Schena, F. and Grandaliano, G. (2010) Therapeutic targeting of classical and lectin pathways of complement protects from ischemia-reperfusion-induced renal damage. Am. J. Pathol. 176, 1648-59.
Cook, H. T. (2013) Complement and kidney disease. Curr. Opin. Nephrol. Hypertens. 22, 295-301.
Degn, S. E., Hansen, A. G., Steffensen, R., Jacobsen, C., Jensenius, J. C. and Thiel, S. (2009) MAp44, a human protein associated with pattern recognition molecules of the complement system and regulating the lectin pathway of complement activation. J. Immunol. 183, 7371-7378.
Degn, S. E., Jensen, L., Hansen, A. G., Duman, D., Tekin, M., Jensenius, J. C. and Thiel, S. (2012) Mannan-binding lectin-associated serine protease (MASP)-1 is crucial for lectin pathway activation in human serum, whereas neither MASP-1 nor MASP-3 is required for alternative pathway function. J. Immunol. 189, 3957-69.
Dobó, J., Harmat, V., Sebestyén, E., Beinrohr, L., Závodszky, P. and Gál, P. (2008) Purification, crystallization and preliminary X-ray analysis of human mannose-binding lectin-associated serine protease-1 (MASP-1) catalytic region. Acta Crystallogr. Sect. F Struct. Biol. Cryst. Commun. 64, 781-784.
Empie, M. W. and Laskowski, M., Jr. (1982) Thermodynamics and kinetics of single residue replacements in avian ovomucoid third domains: effect on inhibitor interactions with serine proteinases. Biochemistry 21, 2274-2284.
Fildes, J. E., Shaw, S. M., Walker, A. H., McAlindon, M., Williams, S. G., Keevil, B. G. and Yonan, N. (2008) Mannose-binding lectin deficiency offers protection from acute graft rejection after heart transplantation. J. Heart Lung Transplant. 27, 1353-1356.
Fujita, T., Matsushita, M. and Endo, Y. (2004) The lectin-complement pathway—its role in innate immunity and evolution. Immunol. Rev. 198, 185-202.
Gál, P., Harmat, V., Kocsis, A., Bián, T., Barna, L., Ambrus, G., Végh, B., Balczer, J., Sim, R. B., Náray-Szabó, G. and Závodszky, P. (2005) A true autoactivating enzyme. Structural insights into mannose-binding lectin-associated serine protease-2 activation. J. Biol. Chem. 280, 33435-33444.
Gál, P., Barna, L., Kocsis, A. and Závodszky P. (2007) Serine proteases of the classical and lectin pathways: Similarities and differences, Immunobiol. 212, 267-277.
Gou, Y., Yang, F., Liang, H. (2016) Designing Prodrugs Based on Special Residues of Human Serum Albumin. Curr. Top. Med. Chem. 16, 996-1008.
Hajela, K., Kojima, M., Ambrus, G., Wong, K. H. N., Moffatt, B. E., Fergula, J., Hajela, S., Gál, P. and Sim, R. B. (2002) The biological functions of MBL-associated serine proteases (MASPs). Immunbiol. 205, 467-475.
Harboe, M and Mollnes, T. E. (2008) The alternative complement pathway revisited. J. Cell. Mol. Med. 12, 1074-1084.
Harmat, V., Gál, P., Kardos, J., Szilágyi, K., Ambrus, G., Végh, B., Náray-Szabó, G. and Závodszky, P. (2004) The structure of MBL-associated serine protease-2 reveals that identical substrate specificities of C1s and MASP-2 are realized through different sets of enzyme-substrate interactions. J. Mol. Biol. 342, 1533-1546.
Héja, D., Kocsis, A., Dobó, J., Szilágyi, K., Szász, R., Závodszky, P., Pál, G. and Gál, P. (2012a) Revised mechanism of complement lectin-pathway activation revealing the role of serine protease MASP-1 as the exclusive activator of MASP-2. Proc. Natl. Acad. Sci. U.S.A 109, 10498-503.
Héja, D., Harmat, V., Fodor, K., Wilmanns, M., Dobó, J., Kékesi, K. A., Závodszky, P., Gál, P., and Pál, G. (2012b) Monospecific inhibitors show that both mannan-binding lectin-associated serine protease-1 (MASP-1) and -2 Are essential for lectin pathway activation and reveal structural plasticity of MASP-2. J. Biol. Chem. 287, 20290-20300.
Ibernon, M., Moreso, F. and Seron, D. (2014) Innate immunity in renal transplantation: the role of mannose-binding lectin. Transplant. Rev. (Orlando.) 28, 21-25.
Ingram, G., Hakobyan, S., Robertson, N. P. and Morgan, B. P. (2009) Complement in multiple sclerosis: its role in disease and potential as a biomarker. Clin. Exp. Immunol. 155, 128-139.
Jameson, G. W., Roberts, D. V., Adams, R. W., Kyle, W. S. A., and Elmore, D. T. (1973) Determination of the operational molarity of solutions of bovine α-chymotrypsin, trypsin, thrombin and factor Xa by spectrofluorimetric titration. Biochem. J. 131, 107-117.
Keizer, M. P., Pouw, R. B., Kamp, A. M., Patiwael, S., Marsman, G., Hart, M. H., Zeerleder, S., Kuijpers, T. W., Wouters, D. (2015) Eur. J. Immunol. 45, 544-50.
Kelly, R. J., Hill, A., Arnold, L. M., Brooksbank, G. L., Richards, S. J., Cullen, M., Mitchell, L. D., Cohen, D. R., Gregory, W. M. and Hillmen, P. (2011) Long-term treatment with eculizumab in paroxysmal nocturnal hemoglobinuria: sustained efficacy and improved survival. Blood. 117, 6786-6792.
Kidmose, R. T., Laursen, N. S., Dobó, J., Kjaer, T. R., Sirotkina, S., Yatime, L., Sottrup-Jensen, L., Thiel, S., Gál, P. and Andersen, G. R. (2012) Structural basis for activation of the complement system by component C4 cleavage. Proc. Natl. Acad. Sci. U.S.A 109, 15425-30.
Kiss, B., Duelli, A., Radnai, L., Kékesi, K. A., Katona, G., and Nyitray, L. (2012) Crystal structure of the S100A4-nonmuscle myosin IIA tail fragment complex reveals an asymmetric target binding mechanism. Proc. Natl. Acad. Sci. U.S.A 109, 6048-6053.
Kocsis, A., Kékesi, K. A., Szász, R., Végh, B. M., Balczer, J., Dobó, J., Závodszky, P., Gál, P. and Pál, G. (2010) Selective Inhibition of the Lectin Pathway of Complement with Phage Display Selected Peptides against Mannose-Binding Lectin-Associated Serine Protease (MASP)-1 and -2: Significant Contribution of MASP-1 to Lectin Pathway Activation. J. Immunol. 185, 4169-4178.
Korsinczky, M. L., Schirra, H. J., Rosengren, K. J., West, J., Condie, B. A., Otvos, L., Anderson, M. A. and Craik, D. J. (2001) Solution structures by 1H NMR of the novel cyclic trypsin inhibitor SFTI-1 from sunflower seeds and an acyclic permutant. J. Mol. Biol. 311, 579-591.
Krarup, A., Gulla, K. C., Gál, P. Hajela, K. and Sim, R. B. (2008) The action of MBL associated serine protease 1 (MASP1) on factor XIII and fibrinogen. Biochim. Biophys. Acta. 1784, 1294-1300.
Kunkel, T. A., Bebenek, K. and Mcclary, J. (1991) Efficient Site-Directed Mutagenesis Using Uracil-Containing DNA. Methods in Enzymology 204, 125-139.
La Bonte, L. R., Pavlov, V. I., Tan, Y. S., Takahashi, K., Takahashi, M., Banda, N. K., Zou, C., Fujita, T. and Stahl, G. L. (2012) Mannose-binding lectin-associated serine protease-1 is a significant contributor to coagulation in a murine model of occlusive thrombosis. J. Immunol. 188:885-91.
Laskowski, M., Jr., and Kato, I. (1980) Protein inhibitors of proteinases. Annu. Rev. Biochem. 49, 593-626.
Lewis, L. A., Ram, S. (2014) Meningococcal disease and the complement system. Virulence. 5, 98-126.
Lobstein, J., Emrich, C. A., Jeans, C., Faulkner, M., Riggs, P., and Berkmen, M. (2012) SHuffle, a novel Escherichia coli protein expression strain capable of correctly folding disulfide bonded proteins in its cytoplasm. Microb. Cell Factories 11, 56.
Luckett, S., Garcia, R. S., Barker, J. J., Konarev, A. V., Shewry, P. R., Clarke, A. R. & Brady, R. L. (1999) High-resolution structure of a potent, cyclic proteinase inhibitor from sunflower seeds. J. Mol. Biol. 290, 525-533.
Malik, Z., Amir, S., Pal, G., Buzás, Z., Várallyay, E., Antal, J., Szilágyi, Z., Vékey, K., Asbóth, B., Patthy, A. and Gráf, L. (1999) Proteinase inhibitors from desert locust, Schistocerca gregaria: engineering of both P-1 and P-1′ residues converts a potent chymotrypsin inhibitor to a potent trypsin. Biochimica Et Biophysica Acta-Protein Structure and Molecular Enzymology 1434, 143-150.
Markiewski, M. M. and Lambris, J. D. (2007) The role of complement in inflammatory diseases—from behind the scenes into the spotlight. Am. J. Pathol. 171, 715-727.
Megyeri M, Makó V, Beinrohr L, Doleschall Z, Prohászka Z, Cervenak L, Závodszky P, Gál P. (2009) Complement protease MASP-1 activates human endothelial cells: PAR4 activation is a link between complement and endothelial function. J. Immunol. 183:3409-16.
Meredith, M. E., Salameh, T. S., Banks, W. A. (2015) Intranasal Delivery of Proteins and Peptides in the Treatment of Neurodegenerative Diseases. AAPS. J. 17, 780-787.
Merle, N. S., Church, S. E., Fremeaux-Bacchi, V., Roumenina, L. T. (2015) Complement System Part I—Molecular Mechanisms of Activation and Regulation. Front. Immunol. 6, 262.
Merle, N. S., Noe, R., Halbwachs-Mecarelli, L., Fremeaux-Bacchi, V. and Roumenina, L. T. (2015) Complement System Part II: Role in Immunity. Front. Immunol. 6, 257.
Mollnes, T. E., Song, W. C. and Lambris, J. D. (2002) Complement in inflammatory tissue damage and disease. Trends Immunol. 23, 61-64.
Morgan, B. P., Marchbank, K. J., Longhi, M. P., Harris, C. L. and Gallimore, A. M. (2005) Complement: central to innate immunity and bridging to adaptive responses. Immunol. Lett. 97, 171-179.
Mulvenna, J. P., Foley, F. M. and Craik, D. J. (2005) Discovery, structural determination, and putative processing of the precursor protein that produces the cyclic trypsin inhibitor sunflower trypsin inhibitor 1. J. Biol. Chem. 280, 32245-32253.
Noris, M. and Remuzzi, G. (2009) Atypical hemolytic-uremic syndrome. N. Engl. J. Med. 361, 1676-1687.
Orsini, F., Chrysanthou, E., Dudler, T., Cummings, W. J., Takahashi, M., Fujita, T., Demopulos, G., De Simoni, M. G. and Schwaeble, W. (2016) Mannan binding lectin-associated serine protease-2 (MASP-2) critically contributes to post-ischemic brain injury independent of MASP-1. J. Neuroinflammation 13, 213.
Ozaki, M., Kang, Y., Tan, Y. S., Pavlov, V. I., Liu, B., Boyle, D. C., Kushak, R. I., Skjoedt, M. O., Grabowski, E. F., Taira, Y. and Stahl, G. L. (2016) Human mannose-binding lectin inhibitor prevents Shiga toxin-induced renal injury. Kidney Int. 90, 774-782.

Pál, G., Kossiakoff, A. A., and Sidhu, S. S. (2003) The functional binding epitope of a high affinity variant of human growth hormone mapped by shotgun alanine-scanning mutagenesis: insights into the mechanisms responsible for improved affinity. J. Mol. Biol. 332, 195-204.
Pál, G., Fong, S.-Y., Kossiakoff, A. A., and Sidhu, S. S. (2005a) Alternative views of functional protein binding epitopes obtained by combinatorial shotgun scanning mutagenesis. Protein Sci. Publ. Protein Soc. 14, 2405-2413.
Pál, G., Ultsch, M. H., Clark, K. P., Currell, B., Kossiakoff, A. A., and Sidhu, S. S. (2005b) Intramolecular cooperativity in a protein binding site assessed by combinatorial shotgun scanning mutagenesis. J. Mol. Biol. 347, 489-494.
Pál, G., Kouadio, J.-L. K., Artis, D. R., Kossiakoff, A. A., and Sidhu, S. S. (2006) Comprehensive and quantitative mapping of energy landscapes for protein-protein interactions by rapid combinatorial scanning. J. Biol. Chem. 281, 22378-22385.
Petersen, L. C., Bjørn, S. E., Olsen, O. H., Nordfang, O., Norris, F., and Norris, K. (1996) Inhibitory properties of separate recombinant Kunitz-type-protease-inhibitor domains from tissue-factor-pathway inhibitor. Eur. J. Biochem. FEBS 235, 310-316.
Petersen, B. H., Lee, T. J., Snyderman, R., Brooks, G. F. (1979) Neisseria meningitidis and Neisseria gonorrhoeae bacteremia associated with C6, C7, or C8 deficiency. Ann. Intern. Med. 90, 917-920.
Petri, C., Thiel, S., Jensenius, J. C. and Herlin, T. (2015) Investigation of Complement-activating Pattern Recognition Molecules and Associated Enzymes as Possible Inflammatory Markers in Oligoarticular and Systemic Juvenile Idiopathic Arthritis. J. Rheumatol. 42, 1252-1258.
Paréj, K., Dobó, J., Závodszky, P. and Gál, P. (2013) The control of the complement lectin pathway activation revisited: both C1-inhibitor and antithrombin are likely physiological inhibitors, while α2-macroglobulin is not. Mol. Immunol. 54, 415-422.
Pavlov, V. I., Skjoedt, M. O., Siow, Tan, Y., Rosbjerg, A., Garred, P. and Stahl, G. L. (2012) Endogenous and natural complement inhibitor attenuates myocardial injury and arterial thrombogenesis. Circulation. 126, 2227-2235.
Pavlov, V. I., Tan, Y. S., McClure, E. E., La Bonte, L. R., Zou, C., Gorsuch, W. B. and Stahl, G. L. (2015) Human mannose-binding lectin inhibitor prevents myocardial injury and arterial thrombogenesis in a novel animal model. Am. J. Pathol. 185, 347-355.
Rapali, P., Radnai, L., Süveges, D., Harmat, V., Tölgyesi, F., Wahlgren, W. Y., Katona, G., Nyitray, L., and Pál, G. (2011) Directed evolution reveals the binding motif preference of the LC8/DYNLL hub protein and predicts large numbers of novel binders in the human proteome. PloS One 6, e18818.
Ricklin D., Hajishengallis G., Yang K. and Lambris J. D. (2010) Complement: a key system for immune surveillance and homeostasis. Nat. Immunol. 11, 785-797.
Schwaeble, W. J., Lynch, N. J., Clark, J. E., Marber, M., Samani, N. J., Ali, Y. M., Dudler, T., Parent, B., Lhotta, K., Wallis, R., Farrar, C. A., Sacks, S., Lee, H., Zhang, M., Iwaki, D., Takahashi, M., Fujita, T., Tedford, C. E. and Stover, C. M. (2011) Targeting of mannan-binding lectin-associated serine protease-2 confers protection from myocardial and gastrointestinal ischemia/reperfusion injury. Proc. Natl. Acad. Sci. U.S.A 108, 7523-7528.
Schechter, I. and Berger, A. (1967) On the size of the active site in proteases. I. Papain. Biochem. Biophys. Res. Commun. 27, 157-162.
Schwertz, H., Carter, J. M., Russ, M., Schubert, S., Schlitt, A., Buerke, U., Schmidt, M., Hillen, H., Werdan, K. and Buerke M. (2008) Serine protease inhibitor nafamostat given before reperfusion reduces inflammatory myocardial injury by complement and neutrophil inhibition. J. Cardiovasc. Pharmacol. 52, 151-160.
Smith, G. P. (1985) Filamentous Fusion Phage—Novel Expression Vectors That Display Cloned Antigens on the Virion Surface. Science 228, 1315-1317.
Szebeni, J. ed. (2004) The complement system. Novel roles in health and disease Kluwer Academic Publishers, ISBN 1-4020-8055-8057.
Szebeni, J. (2005) Complement activation-related pseudoallergy: a new class of drug-induced acute immune toxicity. Toxicology 216, 106-121.
Szenthe, B., Gáspári, Z., Nagy, A., Perczel, A. and Gráf, L. (2004) Same fold with different mobility: backbone dynamics of small protease inhibitors from the desert locust, Schistocerca gregaria. Biochemistry 43, 3376-3384.
Szenthe, B., Patthy, A., Gáspari, Z., Kékesi, A. K., Gráf, L. and Pál, G. (2007) When the surface tells what lies beneath: Combinatorial phage-display mutagenesis reveals complex networks of surface-core interactions in the pacifastin protease inhibitor family. J. Mol. Biol. 370, 63-79.
Takahashi, M., Iwaki, D., Kanno, K., Ishida Y., Xiong, J., Matsushita, M., Endo, Y., Miura, S., Ishii, N., Sugamura, K. and Fujita, T. (2008) Mannose-Binding Lectin of the Lectin Complement Pathway (MASP)-1 Contributes to Activation (MBL)-Associated Serine Protease J. Immunol. 180, 6132-6138.
Takahashi, K., Chang, W-C., Takahashi, M., Pavlov, V., Ishida, Y., La Bonte, L., Shi, L., Fujita, T., Stahl, G. L. and Van Cott, E. M. (2011) Mannose-binding lectin and its associated proteases (MASPs) mediate coagulation and its deficiency is a risk factor in developing complications from infection, including disseminated intravascular coagulation. Immunobiology 216, 96-102
Tichaczek-Goska, D. (2012) Deficiencies and excessive human complement system activation in disorders of multifarious etiology. Adv. Clin. Exp. Med. 21, 105-114.
Tobin, P. H., Richards, D. H., Callender, R. A., Wilson, C. J. (2014) Protein engineering: a new frontier for biological therapeutics. Curr. Drug Metab. 15, 743-756.
Walport, M. J. (2001a) Complement. First of two parts. N. Eng. J. Med. 344, 1058-1066.
Walport, M. J. (2001b) Complement. Second of two parts. N. Eng. J. Med. 344, 1140-1144.
Weiss, G. A., Watanabe, C. K., Zhong, A., Goddard, A. and Sidhu, S. S. (2000) Rapid mapping of protein functional epitopes by combinatorial alanine scanning. Proc. Natl. Acad. Sci. U.S.A 97, 8950-8954.
Wouters, D., Keizer, M., Hart, M., Pouw, R., Wolbink, A., Kuijpers, T., Hamann, D., and Aarden, L. (2011) The lectin pathway is inhibited by an anti-coagulant protease inhibitor acting on MASP-2. Mol. Immunol. 48, 1687.

1. A protein containing any of the sequences according to formula (Ih):
X₁CRX₂X₃X₄X₅ (Ih)
where
X₁ is F, Y, L, P, Q, M, V, W, A, T and
X₂ is A, G, S, T and
X₃ is V, A, I, L, M, D, H, S and
X₄ is K, I, Q, R, H, S, F, M, N, L, V and
X₅ is R, V, I, K, M, Q, E, F, L, N, Y, D, S, H, or
a salt, ester or pharmaceutically acceptable prodrug thereof.

2. The protein according to claim 1, wherein the protein contains any of the following sequences:
(SEQ ID NO: 1)
FCRAVKR,
(SEQ ID NO: 2)
YCRAVKR,
(SEQ ID NO: 3)
LCRAVKR,
(SEQ ID NO: 4)
PCRAVKR,
(SEQ ID NO: 5)
QCRAVKR,
(SEQ ID NO: 6)
MCRAVKR,
(SEQ ID NO: 7)
VCRAVKR,
(SEQ ID NO: 8)
WCRAVKR,
(SEQ ID NO: 9)
PCRALKI
or
(SEQ ID NO: 10)
PCRAVKI
or a salt, ester or pharmaceutically acceptable prodrug thereof.

3. The protein according to claim 2, wherein the protein contains any of the following sequences:
(SEQ ID NO: 1)
FCRAVKR,
or
(SEQ ID NO: 4)
PCRAVKR,
or a salt, ester or pharmaceutically acceptable prodrug thereof.

4. The protein according to claim 1, wherein the protein is a Kunitz domain protein and its sequence tag from position 13 to position 19 according to the position numbering defined for the Kunitz domain in SEQ ID NO: 22 has any of the sequences of formula (Ih), more preferably any of the sequences from SEQ ID NO: 1 to SEQ ID NO: 10, most preferably the sequence of SEQ ID NO: 1 or SEQ ID NO: 4, or a salt, ester or pharmaceutically acceptable prodrug thereof.

5. The protein according to claim 4, wherein the protein is a modified TFPI-D2 protein.

6. The protein according to claim 1 having a sequence sequentially analogous to any of the sequences of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

7. The protein according to claim 6, having a sequence sequentially identical to any of the sequences of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, or SEQ ID NO: 20.

8. A pharmaceutical composition containing one or more proteins, or a salt, ester or pharmaceutically acceptable prodrug thereof according to a claim 1 and at least one pharmaceutically acceptable additive.

9. The pharmaceutical composition according to claim 8, wherein the additive is a matrix ensuring controlled active agent release.

10. The pharmaceutical composition according to claim 8, wherein the pharmaceutical composition is in the form of an infusion, tablet, powder, granule, suppository, injection, syrup, or intranasal delivery system.

11. A kit containing at least one protein, or a salt, ester or pharmaceutically acceptable prodrug thereof according to a claim 1 and one or more additional components.

12. A screening procedure of compounds potentially inhibiting the human MASP-2 enzyme, comprising
i) adding a protein, or a salt, ester or pharmaceutically acceptable prodrug thereof according to a claim 1 in a labelled form to a solution containing said human MASP-2, then
ii) adding a solution containing one or more compounds to be tested, and
iii) measuring the amount of released labelled protein.

13. A method for treating or preventing a disease that is treatable or preventable by inhibiting the complement system, comprising administering to a subject in need thereof an effective amount of the protein, or a salt, ester or pharmaceutically acceptable prodrug thereof according to a claim 1.

14. The method according to claim 13, wherein the disease is selected from the group consisting of (1) ischemia-reperfusion (IR) injuries (especially following recanalyzation after arterial occlusion due to thrombosis or other obstructive diseases), including those occurring after myocardial infarction (e.g. treated by percutaneous coronary interventions or thrombolysis), coronary bypass surgery, organ transplantations, gastrointestinal IR injury, renal IR injury, post-ischemic brain injury, stroke, thrombosis affecting any region of the body; (2) inflammatory and autoimmune conditions with excess activation of the complement system, including autoimmune nephritis (including dense deposit disease, C3 glomerulonephritis), rheumatoid arthritis (RA), juvenile idiopathic arthritis, age-related macular degeneration, systemic lupus erythematosus (SLE), atypical hemolytic uremic syndrome (aHUS), post-infection hemolytic uremic syndrome (HUS), pseudo-allergy developing as a consequence of complement activation (CARPA), paroxysmal nocturnal hemoglobinuria (PNH), polytrauma, graft rejection after organ transplantation; (3) neurodegenerative diseases, preferably Alzheimer's disease, Huntington's disease and Parkinson's diseases and multiple sclerosis.

15-16. (canceled)

17. A process for isolating a human MASP-2 enzyme, comprising
i) contacting a carrier with one or more immobilised proteins, its salt or ester according to claim 1 with a solution containing said human MASP-2 enzyme and
ii) washing the preparation.
